SEQUENCE LISTING (14 sequences)

SEQ ID NO: 1 - DNA, 1143 bp, synthetic construct
  source 1..1143: mol_type = other DNA
atgacgcagaccacccaccacactcccgacaccgcccggcaggccgaccccttcccggtgaagggaatggacgcggtcgtcttcgccgtaggcaacgccaagcaggccgcgcactactactccaccgccttcggcatgcagcttgtggcgtactccggaccggagaacggcagccgcgagaccgcttcgtacgtcctcaccaacggctcggcacgcttcgtcctcacctccgtcatcaagcccgccaccccctggggccacttcctcgccgaccatgtggccgagcacggcgacggcgtcgtcgacctcgccatcgaggtcccggacgcccgcgccgcccacgcgtacgcgatcgagcacggcgcccgctcggtcgccgagccgtacgagctgaaggacgagcacggcacggtcgtcctcgccgcgatcgccacctacggcaagacccgccacaccctcgtcgaccggaccggctacgacggcccctacctccccggctacgtggccgccgccccgatcgtcgaaccgcccgcccaccgcaccttccaggccatcgaccactgcgtcggcaacgtcgagctcggccggatgaacgaatgggtcggcttctacaacaaggtcatgggcttcacgaacatgaaggagttcgtgggcgacgacatcgcgaccgagtactcggcgctgatgtcgaaggtcgtggccgacggcacgctcaaggtcaagttcccgatcaacgagcccgccctcgccaagaagaagtcccagatcgacgagtacctggagttctacggcggcgcgggcgtccagcacatcgcgctgaacacgggtgacatcgtcgagacggtacgcacgatgcgcgccgccggcgtccagttcctggacacgcccgactcgtactacgacaccctcggggagtgggtgggcgacacccgcgtccccgtcgacaccctgcgcgagctgaagatcctcgcggaccgcgacgaggacggctatctgctccagatcttcaccaagccggtccaggaccgcccgacggtcttcttcgagatcatcgaacgccacggctcgatgggattcggcaagggcaacttcaaggccctgttcgaggcgatcgagcgggagcaggagaagcggggcaacctg

SEQ ID NO: 2 - protein, 381 aa, synthetic construct
MTQTTHHTPDTARQADPFPVKGMDAVVFAVGNAKQAAHYYSTAFGMQLVAYSGPENGSRETASYVLTNGSARFVLTSVIKPATPWGHFLADHVAEHGDGVVDLAIEVPDARAAHAYAIEHGARSVAEPYELKDEHGTVVLAAIATYGKTRHTLVDRTGYDGPYLPGYVAAAPIVEPPAHRTFQAIDHCVGNVELGRMNEWVGFYNKVMGFTNMKEFVGDDIATEYSALMSKVVADGTLKVKFPINEPALAKKKSQIDEYLEFYGGAGVQHIALNTGDIVETVRTMRAAGVQFLDTPDSYYDTLGEWVGDTRVPVDTLRELKILADRDEDGYLLQIFTKPVQDRPTVFFEIIERHGSMGFGKGNFKALFEAIEREQEKRGNL

SEQ ID NO: 3 - DNA, 1072 bp, synthetic construct
  source 1..1072: mol_type = other DNA
atgcagaatttcgagatcgactacgtagaaatgtatgtggaaaatcttgaggtggctgcgttcagctgggtggacaagtacgcgttcgccgtcgccggtaccagccggtcggcggaccaccggagcatcgcgctacggcaggggcaggtgacgctggtcctcaccgagcccacgtcggatcgtcacccggcggcggcctacctccagacgcacggcgacggtgtcgccgacatcgcgatggccacttcggacgtcgccgccgcttacgaggccgcggtgcgggcgggggcggaagccgtccgcgcgccggggcagcactcggaggccgcggtcacgacggccaccatcggtggtttcggcgatgtcgtgcacaccctgattcagcgcgacggaacatccgccgagctgcccccgggcttcaccggctcgatggacgtcaccaaccacggcaagggagatgtcgacctgctcggcatcgaccacttcgcgatctgcctgaacgccggcgacctcggccccacggtggagtactacgagcgggcactgggtttccggcagatcttcgacgagcacatcgtggtcggtgcgcaggcgatgaactccaccgtcgtgcagagtgcgtccggagcggtcaccctcaccctgatcgagcccgaccggaacgccgaccccggccagatcgacgagttcctcaaggatcaccaaggggcaggcgttcagcacatcgccttcaacagcaacgacgcggtccgtgcggtcaaggcgctgtccgagcgcggggtggagttcctcaagacgccgggggcctactacgacctgctcggtgagcggatcacgctgcagacgcactcgctggacgatctgcgggcgacgaacgtgctcgccgatgaggaccacggcggccagctgttccagatcttcaccgcgtccactcatccgcgtcacacgattttcttcgaggtcatcgagcggcagggcgcgggcaccttcggcagctccaacatcaaggccctgtacgaggccgttgagctggaacgcaccgggcagagcgagttcggcgccgctcggcgat

SEQ ID NO: 4 - protein, 357 aa, synthetic construct
MQNFEIDYVEMYVENLEVAAFSWVDKYAFAVAGTSRSADHRSIALRQGQVTLVLTEPTSDRHPAAAYLQTHGDGVADIAMATSDVAAAYEAAVRAGAEAVRAPGQHSEAAVTTATIGGFGDVVHTLIQRDGTSAELPPGFTGSMDVTNHGKGDVDLLGIDHFAICLNAGDLGPTVEYYERALGFRQIFDEHIVVGAQAMNSTVVQSASGAVTLTLIEPDRNADPGQIDEFLKDHQGAGVQHIAFNSNDAVRAVKALSERGVEFLKTPGAYYDLLGERITLQTHSLDDLRATNVLADEDHGGQLFQIFTASTHPRHTIFFEVIERQGAGTFGSSNIKALYEAVELERTGQSEFGAARR

SEQ ID NO: 5 - DNA, 1521 bp, synthetic construct
  source 1..1521: mol_type = other DNA
atgaatacaattaacgaatatttaagtttagaagaatttgaggccataatctttggaaatcaaaaagttacaattagcgacgttgtcgtaaaccgagttaatgaaagtttcaatttcttaaaagaattctctgggaataaagtaatttacggcgtaaacactggttttggaccaatggcgcagtatagaattaaagagtctgatcaaattcagcttcaatataatttaattagaagccactcttcgggaaccggaaagccattaagcccggtttgtgctaaagcagcaattttagctcgtttaaacactctttcattaggaaattcaggcgttcacccgtcagtaatcaacctaatgtctgagcttatcaataaagatattacccctttaatcttcgaacacggaggcgttggtgccagcggtgacttagtacagctgtcacatttagctttagtgttaattggcgaaggtgaagttttttataaaggtgaaagaagaccaactcctgaagtttttgaaattgaaggtttaaaaccaattcaggtagaaattagagaaggtctggcattaattaacggaacttcagtaatgactggaattggagttgtaaatgtttatcatgctaaaaaattattagactggtcattaaaatcttcttgtgcaattaacgaattggttcaggcttatgacgatcatttctcagcagaactaaaccagactaaacgtcataaaggacagcaggaaattgcgttgaaaatgcgtcaaaatctttcagacagtactttaatccgtaaaagagaagatcatttatattctggtgaaaataccgaagaaatcttcaaagaaaaagttcaggaatattattcattaagatgcgttcctcaaattttaggaccggttttagaaactattaataatgtagcttctattctggaagacgaatttaactcggcaaacgataacccaattatcgatgtaaaaaaccagcatgtttaccacggcggaaatttccacggagattatatttcgcttgaaatggataaactaaaaattgttattaccaaattaacaatgctggcagaacgtcagttgaactatttattaaattcaaaaatcaacgaattacttcctccgtttgtaaacttaggaacattaggatttaatttcggaatgcagggtgttcagtttacagcaacatctacaacggcagaaagtcaaatgctgtcgaacccaatgtatgttcacagtattccaaacaacaacgacaatcaggatatcgtaagtatgggaactaactcggcagtaattacctcaaaagtaatcgaaaatgcttttgaagttctggctatcgaaatgattactatcgttcaggcaattgattatttaggacaaaaagacaaaatttcatctgtttctaaaaaatggtacgatgaaattagaaatataattccaacatttaaggaagatcaggtaatgtatcctttcgttcaaaaagtaaaagatcatttgatcaacaattaa

SEQ ID NO: 6 - protein, 506 aa, synthetic construct
MNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 7 - DNA, 1188 bp, synthetic construct
  source 1..1188: mol_type = other DNA
atggccatgaccacctatagcgataaaggcgcgaaaccggaacgcggccgctttctgcattttcatagcgtgaccttttgggtgggcaacgcgaaacaagcggcgagcttttattgcagcaaaatgggctttgaaccgctggcgtatcgcggactggaaaccggcagccgcgaagtggtgagccatgtgattaaacaaggcaaaattgtgtttgtgctgagcagcgcgctgaacccgtggaacaaagaaatgggcgatcatctggtgaaacatggcgatggcgtgaaagatattgcgtttgaagtggaagattgcgattatattgtgcagaaagcgcgtgaacgcggcgcgaaaattatgcgcgaaccgtgggtggaacaagataaatttggcaaagtgaaatttgcggtgctgcagacctatggcgataccacccataccctggtggaaaaaatgaactatattggtcagtttctgccgggctatgaagcgccggcgtttatggacccgctgctgccgaaactgccgaaatgcagcctggaaatgattgatcatattgtgggcaatcagccggatcaagaaatggtgagcgcgagcgaatggtatctgaaaaacctgcagtttcatcgcttttggagcgtggatgatacccaagtgcataccgaatatagcagcctgcgcagcattgtggtggcgaactatgaagaaagcattaaaatgccgattaacgaaccggcgccgggcaaaaaaaaaagtcagattcaagaatatgtggattataacggcggcgcgggcgtgcagcatattgcgctgaaaaccgaagatattattaccgcgattcgccatctgagagaacgcggcctggaatttctgagcgtgccgagcacctattataaacagctgcgcgaaaaactgaaaaccgcgaaaattaaagtgaaagaaaacattgatgcgctggaagaactgaaaattctggtggattatgatgaaaaaggctatctgctgcagatttttaccaaaccggtgcaagatcgcccgaccctgtttctggaagtgattcagcgccataaccatcaaggctttggcgcgggcaactttaacagcctgtttaaagcgtttgaagaagaacagaacctgcgcggcaacctgaccaacatggaaaccaacggcgtggtgccgggcatgtaa

SEQ ID NO: 8 - protein, 393 aa, synthetic construct
MTTYSDKGAKPERGRFLHFHSVTFWVGNAKQAASFYCSKMGFEPLAYRGLETGSREVVSHVIKQGKIVFVLSSALNPWNKEMGDHLVKHGDGVKDIAFEVEDCDYIVQKARERGAKIMREPWVEQDKFGKVKFAVLQTYGDTTHTLVEKMNYIGQFLPGYEAPAFMDPLLPKLPKCSLEMIDHIVGNQPDQEMVSASEWYLKNLQFHRFWSVDDTQVHTEYSSLRSIVVANYEESIKMPINEPAPGKKKSQIQEYVDYNGGAGVQHIALKTEDIITAIRHLRERGLEFLSVPSTYYKQLREKLKTAKIKVKENIDALEELKILVDYDEKGYLLQIFTKPVQDRPTLFLEVIQRHNHQGFGAGNFNSLFKAFEEEQNLRGNLTNMETNGVVPGM

SEQ ID NO: 9 - DNA, 1521 bp, synthetic construct
  source 1..1521: mol_type = other DNA
atgattgcggataaaagcattaacctggataccctgcataaagtgctgtttgataacgaaaaactgaaactgagcgaagaatgcattcgcaaagtggaagaaagctttgattttctgcagagctttagcagcgataaaattatttatggcattaacaccggctttggcccgatggcgcagtatcgcattgaagatcagagcctgattgatctgcagtataacattattcgcagccatagcaccggcgcgggcaaaccgctgccggaactgtatgtgaaagcggcgatgattgcgcgcctgtatacctttctgcaaggcaaaagcggcgtgcatctggaactggtgagcctgctgtgcgaatttattaaccgcggcatttatccgtttattccggaacatggcagcgtgggcgcgagcggcgatctggtgcagctggcgcatattgcgctgaccctgattggcgaaggcgaagtgttttatcaaggcaaactgtgcaacgcggcgaccgtgctgcaagaaaacggcctgaaaccgtttagcatgcgcattcgcgaaggcctgagcgtgaccaacggcacgagcgtgatgaccggcattggcattgtgaacctgatttatgcgaaaaaactgctgcgctggagcgtggcggcgagcgtgatgatgaacgaaattgcagcgtcatacgatgattttatggctcaagcgttgaacgaagcgaaacatcataaaggtcagcaagaaattgcggcgatgatgcgcgaatgggtggcgggcagcaaaagcgtgctgcagcgcgaaaacgaactgtataaccaagtgcataaagaaaaaatttttgaacataaagtgcagccgtattatagcctgcgctgcgtgccgcagattctgggcccgatttatgatgaactggaaaacgcggaagaagtgctgattaacgaaattaacagcgcgtgcgataacccgattgttgatccggatacgcagaacatttatcatggcggcaactttcatggcgattatattagctttgaaatggataaactgaaaattgcggtgaccaaactgaccatgctgtgcgaacgtcagattaactatctgtttcatgatcgcattaacggcattctgccgccgtttgtgaacctgggcgtgctgggcctgaactatggcctgcaagcgagtcagtttaccgcgacgagcaccaccgcggaatgtcagaccctgagcaacccgatgtatgtgcatagcattccgaacaacaacgataaccaagatattgtgagcatgggcaccaacagcgcgctgctggcgaaaaccgtgattgaaaacagctatcaagtgatggcgattcagtttatgggcatggcgcaagcgatagattacctgaaaatccaagatcgcctgagcagcaaaagccgccaagtgtatgaagaaattcgcagcttttttccggtgtttaccaacgataccccgaaatataaagaaattgaaatgatgattgattacctgaagaaggaggataaa

SEQ ID NO: 10 - protein, 507 aa, synthetic construct
MIADKSINLDTLHKVLFDNEKLKLSEECIRKVEESFDFLQSFSSDKIIYGINTGFGPMAQYRIEDQSLIDLQYNIIRSHSTGAGKPLPELYVKAAMIARLYTFLQGKSGVHLELVSLLCEFINRGIYPFIPEHGSVGASGDLVQLAHIALTLIGEGEVFYQGKLCNAATVLQENGLKPFSMRIREGLSVTNGTSVMTGIGIVNLIYAKKLLRWSVAASVMMNEIAASYDDFMAQALNEAKHHKGQQEIAAMMREWVAGSKSVLQRENELYNQVHKEKIFEHKVQPYYSLRCVPQILGPIYDELENAEEVLINEINSACDNPIVDPDTQNIYHGGNFHGDYISFEMDKLKIAVTKLTMLCERQINYLFHDRINGILPPFVNLGVLGLNYGLQASQFTATSTTAECQTLSNPMYVHSIPNNNDNQDIVSMGTNSALLAKTVIENSYQVMAIQFMGMAQAIDYLKIQDRLSSKSRQVYEEIRSFFPVFTNDTPKYKEIEMMIDYLKKEDK

SEQ ID NO: 11 - DNA, 4485 bp, synthetic construct
  source 1..4485: mol_type = other DNA
agttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctagaaataattttgtttaactttaagaaggagatatacatatgacgcagaccacccaccacactcccgacaccgcccggcaggccgaccccttcccggtgaagggaatggacgcggtcgtcttcgccgtaggcaacgccaagcaggccgcgcactactactccaccgccttcggcatgcagcttgtggcgtactccggaccggagaacggcagccgcgagaccgcttcgtacgtcctcaccaacggctcggcacgcttcgtcctcacctccgtcatcaagcccgccaccccctggggccacttcctcgccgaccatgtggccgagcacggcgacggcgtcgtcgacctcgccatcgaggtcccggacgcccgcgccgcccacgcgtacgcgatcgagcacggcgcccgctcggtcgccgagccgtacgagctgaaggacgagcacggcacggtcgtcctcgccgcgatcgccacctacggcaagacccgccacaccctcgtcgaccggaccggctacgacggcccctacctccccggctacgtggccgccgccccgatcgtcgaaccgcccgcccaccgcaccttccaggccatcgaccactgcgtcggcaacgtcgagctcggccggatgaacgaatgggtcggcttctacaacaaggtcatgggcttcacgaacatgaaggagttcgtgggcgacgacatcgcgaccgagtactcggcgctgatgtcgaaggtcgtggccgacggcacgctcaaggtcaagttcccgatcaacgagcccgccctcgccaagaagaagtcccagatcgacgagtacctggagttctacggcggcgcgggcgtccagcacatcgcgctgaacacgggtgacatcgtcgagacggtacgcacgatgcgcgccgccggcgtccagttcctggacacgcccgactcgtactacgacaccctcggggagtgggtgggcgacacccgcgtccccgtcgacaccctgcgcgagctgaagatcctcgcggaccgcgacgaggacggctatctgctccagatcttcaccaagccggtccaggaccgcccgacggtcttcttcgagatcatcgaacgccacggctcgatgggattcggcaagggcaacttcaaggccctgttcgaggcgatcgagcgggagcaggagaagcggggcaacctgtaggcggcgcggcccgggtaccgagctcgaattcgccctatagtgagtcgtattacaattcactggccgtcgttttacaacgtcgtgactgggctgcaggatccactagtaacggccgccagtgtgctggaattctgcagatatccatcacactggcggccgctcgagcagatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatc

SEQ ID NO: 12 - DNA, 4254 bp, synthetic construct
  source 1..4254: mol_type = other DNA
agtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctagaaataattttgtttaactttaagaaggagatatacatatgcagaatttcgagatcgactacgtagaaatgtatgtggaaaatcttgaggtggctgcgttcagctgggtggacaagtacgcgttcgccgtcgccggtaccagccggtcggcggaccaccggagcatcgcgctacggcaggggcaggtgacgctggtcctcaccgagcccacgtcggatcgtcacccggcggcggcctacctccagacgcacggcgacggtgtcgccgacatcgcgatggccacttcggacgtcgccgccgcttacgaggccgcggtgcgggcgggggcggaagccgtccgcgcgccggggcagcactcggaggccgcggtcacgacggccaccatcggtggtttcggcgatgtcgtgcacaccctgattcagcgcgacggaacatccgccgagctgcccccgggcttcaccggctcgatggacgtcaccaaccacggcaagggagatgtcgacctgctcggcatcgaccacttcgcgatctgcctgaacgccggcgacctcggccccacggtggagtactacgagcgggcactgggtttccggcagatcttcgacgagcacatcgtggtcggtgcgcaggcgatgaactccaccgtcgtgcagagtgcgtccggagcggtcaccctcaccctgatcgagcccgaccggaacgccgaccccggccagatcgacgagttcctcaaggatcaccaaggggcaggcgttcagcacatcgccttcaacagcaacgacgcggtccgtgcggtcaaggcgctgtccgagcgcggggtggagttcctcaagacgccgggggcctactacgacctgctcggtgagcggatcacgctgcagacgcactcgctggacgatctgcgggcgacgaacgtgctcgccgatgaggaccacggcggccagctgttccagatcttcaccgcgtccactcatccgcgtcacacgattttcttcgaggtcatcgagcggcagggcgcgggcaccttcggcagctccaacatcaaggccctgtacgaggccgttgagctggaacgcaccgggcagagcgagttcggcgccgctcggcgatgactcgagcagatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacg

SEQ ID NO: 13 - DNA, 6457 bp, synthetic construct
  source 1..6457: mol_type = other DNA
aaaaaaaaaagtcagattcaagaatatgtggattataacggcggcgcgggcgtgcagcatattgcgctgaaaaccgaagatattattaccgcgattcgccatctgagagaacgcggcctggaatttctgagcgtgccgagcacctattataaacagctgcgcgaaaaactgaaaaccgcgaaaattaaagtgaaagaaaacattgatgcgctggaagaactgaaaattctggtggattatgatgaaaaaggctatctgctgcagatttttaccaaaccggtgcaagatcgcccgaccctgtttctggaagtgattcagcgccataaccatcaaggctttggcgcgggcaactttaacagcctgtttaaagcgtttgaagaagaacagaacctgcgcggcaacctgaccaacatggaaaccaacggcgtggtgccgggcatgtaaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatggccatgaccacctatagcgataaaggcgcgaaaccggaacgcggccgctttctgcattttcatagcgtgaccttttgggtgggcaacgcgaaacaagcggcgagcttttattgcagcaaaatgggctttgaaccgctggcgtatcgcggactggaaaccggcagccgcgaagtggtgagccatgtgattaaacaaggcaaaattgtgtttgtgctgagcagcgcgctgaacccgtggaacaaagaaatgggcgatcatctggtgaaacatggcgatggcgtgaaagatattgcgtttgaagtggaagattgcgattatattgtgcagaaagcgcgtgaacgcggcgcgaaaattatgcgcgaaccgtgggtggaacaagataaatttggcaaagtgaaatttgcggtgctgcagacctatggcgataccacccataccctggtggaaaaaatgaactatattggtcagtttctgccgggctatgaagcgccggcgtttatggacccgctgctgccgaaactgccgaaatgcagcctggaaatgattgatcatattgtgggcaatcagccggatcaagaaatggtgagcgcgagcgaatggtatctgaaaaacctgcagtttcatcgcttttggagcgtggatgatacccaagtgcataccgaatatagcagcctgcgcagcattgtggtggcgaactatgaagaaagcattaaaatgccgattaacgaaccggcgccgggc

SEQ ID NO: 14 - DNA, 6853 bp, synthetic construct
  source 1..6853: mol_type = other DNA
aaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgattgcggataaaagcattaacctggataccctgcataaagtgctgtttgataacgaaaaactgaaactgagcgaagaatgcattcgcaaagtggaagaaagctttgattttctgcagagctttagcagcgataaaattatttatggcattaacaccggctttggcccgatggcgcagtatcgcattgaagatcagagcctgattgatctgcagtataacattattcgcagccatagcaccggcgcgggcaaaccgctgccggaactgtatgtgaaagcggcgatgattgcgcgcctgtatacctttctgcaaggcaaaagcggcgtgcatctggaactggtgagcctgctgtgcgaatttattaaccgcggcatttatccgtttattccggaacatggcagcgtgggcgcgagcggcgatctggtgcagctggcgcatattgcgctgaccctgattggcgaaggcgaagtgttttatcaaggcaaactgtgcaacgcggcgaccgtgctgcaagaaaacggcctgaaaccgtttagcatgcgcattcgcgaaggcctgagcgtgaccaacggcacgagcgtgatgaccggcattggcattgtgaacctgatttatgcgaaaaaactgctgcgctggagcgtggcggcgagcgtgatgatgaacgaaattgcagcgtcatacgatgattttatggctcaagcgttgaacgaagcgaaacatcataaaggtcagcaagaaattgcggcgatgatgcgcgaatgggtggcgggcagcaaaagcgtgctgcagcgcgaaaacgaactgtataaccaagtgcataaagaaaaaatttttgaacataaagtgcagccgtattatagcctgcgctgcgtgccgcagattctgggcccgatttatgatgaactggaaaacgcggaagaagtgctgattaacgaaattaacagcgcgtgcgataacccgattgttgatccggatacgcagaacatttatcatggcggcaactttcatggcgattatattagctttgaaatggataaactgaaaattgcggtgaccaaactgaccatgctgtgcgaacgtcagattaactatctgtttcatgatcgcattaacggcattctgccgccgtttgtgaacctgggcgtgctgggcctgaactatggcctgcaagcgagtcagtttaccgcgacgagcaccaccgcggaatgtcagaccctgagcaacccgatgtatgtgcatagcattccgaacaacaacgataaccaagatattgtgagcatgggcaccaacagcgcgctgctggcgaaaaccgtgattgaaaacagctatcaagtgatggcgattcagtttatgggcatggcgcaagcgatagattacctgaaaatccaagatcgcctgagcagcaaaagccgccaagtgtatgaagaaattcgcagcttttttccggtgtttaccaacgataccccgaaatataaagaaattgaaatgatgattgattacctgaagaaggaggataaataaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaac

(19) United States
(12) Patent Application Publication
(10) Pub. No.: US 2026/0258393 A1
(43) Pub. Date: Sep. 3, 2026

(54) METHODS FOR CREATING COMPETITIONS FOR TYROSINE BREAKDOWN IN SYNTHETIC MICROBIAL COMMUNITIES AND BETWEEN A GUT MICROBIAL PATHWAY AND A HUMAN PATHWAY

(71) Applicant: Dhara D. SHAH, Chandler, AZ (US)

(72) Inventor: Dhara D. SHAH, Chandler, AZ (US)

(21) Appl. No.: 19/551,882

(22) Filed: Feb. 27, 2026

Related U.S. Application Data
(60) Provisional application No. 63/764,986, filed on Feb. 28, 2025.

(51) Int. Cl. C12N 9/88; C12P 7/42; C12P 7/62

(52) U.S. Cl. CPC C12N 9/88; C12P 7/42; C12P 7/62

(57) ABSTRACT

Described herein is a method for characterizing competition for tyrosine metabolism in a synthetic microbial community that includes providing two or three microbial strains, each expressing a different enzyme that converts tyrosine or a tyrosine pathway intermediate into distinct metabolites. Additionally, a metabolic diversion of the human tyrosine breakdown pathway facilitated by a gut microbial enzyme is described. Diverting tyrosine metabolism allows for the measurement of the resulting metabolites and the characterization of tyrosine metabolism in synthetic microbial communities and provides a system to mimic tyrosine metabolism due to competition between a human pathway and a gut microbial pathway.

### CROSS-REFERENCE TO RELATED APPLICATIONS

This application claims priority to U.S. Provisional Patent Application No. 63/764,986, filed on Feb. 28, 2025, which is incorporated by reference herein in its entirety.

### REFERENCE TO SEQUENCE LISTING

This application was filed with a Sequence Listing XML in ST.26 XML format in accordance with 37 C.F.R. § 1.831. The Sequence Listing XML file submitted in the USPTO Patent Center, “208192-0039-US02_sequence_listing_xml_19 Feb. 2026.xml,” was created on Feb. 19, 2025, contains 14 sequences, has a file size of 42.7 kilobytes (43,806 bytes), and is incorporated by reference in its entirety into the specification.

### BACKGROUND

Tyrosine is a proteinogenic amino acid that undergoes diverse metabolic transformations in both aerobic and anaerobic organisms. In aerobic organisms, oxidative tyrosine catabolism involves a multi-step pathway that produces fumarate and acetoacetate, which can enter the tricarboxylic acid cycle and serve as energy sources. Various enzymes participate in tyrosine metabolism, including tyrosine aminotransferase, 4-hydroxyphenylpyruvate dioxygenase, hydroxymandelate synthase, and tyrosine ammonia lyase, each directing tyrosine or its intermediates toward different metabolic products. When multiple tyrosine breakdown pathways are present within the same environment, the abundances of metabolites can be influenced by direct competition for substrate between enzymes of these metabolic pathways. Defects in the human tyrosine breakdown pathway can lead to metabolic disorders such as tyrosinemias, hawkinsinuria, and alkaptonuria, which result from accumulation of harmful intermediates or products due to mutations or abnormal activities in one or more enzymes involved in tyrosine catabolism.

Current therapeutic approaches for addressing metabolic disorders associated with tyrosine breakdown have focused on inhibiting enzymes within the catabolic pathway. For example, existing therapies for type I tyrosinemia involve administration of compounds that inhibit 4-hydroxyphenylpyruvate dioxygenase, which catalyzes an early step in the tyrosine catabolic pathway. However, because the first reaction in the pathway is reversible, such inhibition can lead to elevated blood tyrosine concentrations, which may result in physiological abnormalities over time due to the relatively low solubility of tyrosine. Alternative strategies that could divert tyrosine metabolism away from pathways producing harmful metabolites while maintaining continuous utilization of tyrosine would be beneficial for developing improved therapeutic interventions.

What is needed are compositions and methods for addressing metabolic disorders associated with tyrosine catabolism.

### SUMMARY

One embodiment described herein is a method for analyzing competition for tyrosine breakdown, comprising: providing a first microbial strain expressing a first enzyme that converts a tyrosine pathway intermediate to a first metabolite; providing a second microbial strain expressing a second enzyme that converts the tyrosine pathway intermediate to a second metabolite different from the first metabolite; optionally providing a third microbial strain expressing a third enzyme that converts tyrosine to a third metabolite different from the first metabolite and the second metabolite; combining the first microbial strain, the second microbial strain, and the third microbial strain, if present, to form a synthetic microbial community; culturing the synthetic microbial community in a medium comprising tyrosine, thereby creating competition between the first enzyme, the second enzyme, and the third enzyme, if present. In one aspect, the method further comprises: measuring a quantity of the first metabolite, the second metabolite, or the third metabolite, wherein the first enzyme, the second enzyme, and the third enzyme, if present, compete for the tyrosine pathway intermediate or tyrosine, thereby diverting tyrosine metabolism from a first pathway producing the first metabolite toward a second pathway producing the second metabolite or a third pathway producing the third metabolite. In another aspect, the first microbial strain, the second microbial strain, and the third microbial strain, if present, are Escherichia coli strains. In another aspect, the Escherichia coli strains are E. coli BL21 (DE3) strains. In another aspect, the tyrosine pathway intermediate is 4-hydroxyphenylpyruvate. In another aspect, the first enzyme is a 4-hydroxyphenylpyruvate dioxygenase that converts 4-hydroxyphenylpyruvate to homogentisate. In another aspect, the 4-hydroxyphenylpyruvate dioxygenase is from Streptomyces avermitilis or Homo sapiens. In another aspect, the second enzyme is a hydroxymandelate synthase that converts 4-hydroxyphenylpyruvate to hydroxymandelate. In another aspect, the hydroxymandelate synthase is from Amycolatopsis orientalis. In another aspect, the second enzyme is a tyrosine ammonia lyase that converts tyrosine to p-coumaric acid or the third enzyme is a tyrosine ammonia lyase that converts tyrosine to p-coumaric acid. In another aspect, the tyrosine ammonia lyase is from Flavobacterium johnsoniae. In another aspect, the tyrosine ammonia lyase is from Bacteroides ovatus. In another aspect, the first microbial strain and the second microbial strain are combined at a ratio between about 1:4 and about 4:1. In another aspect, measuring a quantity of the first metabolite, the second metabolite, or the third metabolite comprises: centrifuging the medium to form a supernatant and a pellet; and measuring an absorbance of the supernatant, wherein the first metabolite is pyomelanin.

Another embodiment described herein is a composition comprising: a first recombinant Escherichia coli strain comprising an expression vector encoding a 4-hydroxyphenylpyruvate dioxygenase capable of converting 4-hydroxyphenylpyruvate (HPP) into homogentisate (HG), wherein the first recombinant Escherichia coli strain produces pyomelanin by secreting HG; a second recombinant Escherichia coli strain comprising an expression vector encoding a second enzyme that converts a tyrosine pathway intermediate to a second metabolite; and optionally a third recombinant Escherichia coli strain comprising an expression vector encoding a third enzyme that converts tyrosine to a third metabolite different from the first and second metabolites; wherein the first recombinant Escherichia coli strain produces a quantity of pyomelanin inversely proportional to an activity of the second enzyme. In one aspect, the first enzyme is a 4-hydroxyphenylpyruvate dioxygenase from Streptomyces avermitilis or a 4-hydroxyphenylpyruvate dioxygenase from Homo sapiens. In another aspect, the second enzyme is a hydroxymandelate synthase from Amycolatopsis orientalis or a tyrosine ammonia lyase from Bacteroides ovatus. In another aspect, the third enzyme is a tyrosine ammonia lyase from Flavobacterium johnsoniae. In another aspect, the first recombinant Escherichia coli strain, the second recombinant Escherichia coli strain, and the third recombinant Escherichia coli strain, if present, are E. coli BL21 (DE3) strains. In another aspect, the ratio of the first recombinant Escherichia coli strain to the second recombinant Escherichia coli strain is between about 1:4 and about 4:1.

Another embodiment described herein is a method for detecting a concentration of one or more tyrosine pathway intermediates in a sample, the method comprising: administering to the sample one or more bacterial strains comprising an expression vector encoding an enzyme selected from the group consisting of: a 4-hydroxyphenylpyruvate dioxygenase from Streptomyces avermitilis or from Homo sapiens; a hydroxymandelate synthase from Amycolatopsis orientalis; and a tyrosine ammonia lyase from Bacteroides ovatus or Flavobacterium johnsoniae; centrifuging the sample to isolate a supernatant; and measuring the absorbance of the supernatant to determine a concentration of pyomelanin. In one aspect, measuring the absorbance of the supernatant comprises analyzing the supernatant via HPLC.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows tyrosine catabolism pathway from aerobic organisms. In the pathway, tyrosine is converted to fumarate and acetoacetate with five successive steps.

FIG. 2A-C show pyomelanin serves as the indicator for secreted homogentisate. FIG. 2A-B show pigment production in cultures of E. coli BL21 (DE3) harboring SaHPPD (FIG. 2A) under four different conditions (FIG. 2B). FIG. 2C shows quantification of pyomelanin production was carried out by normalizing pyomelanin absorbance by cell density for each strain in four different conditions. ANOVA test was used to calculate statistical significance and p-values, n=3.

FIG. 3 shows tyrosine catabolism pathway diversion with one enzyme. The figure depicts the possible route for redirecting the tyrosine breakdown pathway by adding one additional enzyme involved in tyrosine metabolism. Here, hydroxymandelate synthase (HMS) can convert tyrosine to hydroxymandelate (HMA).

FIG. 4 shows the generation of E. coli strains for Tyrosine catabolism pathway diversion with one enzyme. The figure depicts E. coli strains designed for redirecting the tyrosine breakdown pathway by adding one additional enzyme involved in tyrosine metabolism. Here, hydroxymandelate synthase (HMS) can convert tyrosine to hydroxymandelate (HMA).

FIG. 5A-B show creating a competition between E. coli strains harboring SaHPPD and AoHMS. FIG. 5A shows competition for tyrosine breakdown was developed using two environmental microbial enzymes, AoHMS and SaHPPD in a mixed synthetic community of E. coli strains. The spent media from cultures of E. coli BL21 (DE3) harboring AoHMS and SaHPPD in various ratios (1:1, 3:2, and 4:1) while induced with 1 mM IPTG and in the presence of 2 mM tyrosine showed pigment production. FIG. 5B shows the quantification of pyomelanin production normalized by cell density for each sample is depicted in graphical format where ANOVA test was used to calculate statistical significance and p values, n=3. The p-values for this graph indicate the significant or non-significant differences of pyomelanin produced in comparison to the pigment production in the E. coli culture harboring SaHPPD.

FIG. 6 shows the quantification of metabolites from competition between E. coli strains harboring SaHPPD and AoHMS. The concentrations of secreted metabolites within the spent media of synthetic communities harboring SaHPPD and AoHMS at various ratios were measured. Each grey region represents an individual sample, while the different colors in each grey region represent separate metabolites. ANOVA test was used to calculate statistical significance and p values, n=3. The p-values show the significance between HG production in samples where SaHPPD is in competition with AoHMS, compared to when SaHPPD is alone.

FIG. 7 shows the tyrosine catabolism pathway diversion with two additional enzymes. The figure depicts the possible route for redirecting the tyrosine breakdown pathway by adding two additional enzymes involved in tyrosine metabolism from environmental microbes. Here, hydroxymandelate synthase (HMS) can convert tyrosine to hydroxymandelate (HMA) and tyrosine ammonia lyase (TAL) converts tyrosine to p-coumaric acid (pCA).

FIG. 8 shows generation of E. coli strains for tyrosine catabolism pathway diversion with two additional enzymes. The figure depicts E. coli strains designed for redirecting the tyrosine breakdown pathway by adding two additional enzymes involved in tyrosine metabolism from environmental microbes. Here, hydroxymandelate synthase (HMS) can convert tyrosine to hydroxymandelate (HMA) and tyrosine ammonia lyase (TAL) converts tyrosine to p-coumaric acid (pCA).

FIG. 9 shows the quantification of metabolites from competition between E. coli strains harboring SaHPPD, AoHMS, and FjTAL. The concentrations of secreted metabolites within the spent media of synthetic communities harboring SaHPPD, AoHMS, and FjTAL at various ratios were measured. Each grey region represents an individual sample, while the different colors in each grey region represent separate metabolites. ANOVA test was used to calculate statistical significance and p-values, n=3. The p-values show the significance between HG production in samples where SaHPPD is in competition with AoHMS and/or FjTAL, compared to the individual culture of SaHPPD.

FIG. 10 shows a possible mammalian tyrosine catabolism pathway diversion with a gut microbial enzyme. The figure depicts the possible route for redirecting the mammalian tyrosine breakdown pathway by adding a gut microbial enzyme involved in the tyrosine metabolism. Here, a gut microbial tyrosine ammonia lyase (TAL) converts tyrosine to p-coumaric acid (pCA).

FIG. 11 shows the generation of E. coli strains for mammalian tyrosine catabolism pathway diversion with a gut microbial enzyme. The figure depicts E. coli strains designed for redirecting the mammalian tyrosine breakdown pathway by adding a gut microbial enzyme involved in the tyrosine metabolism. Here, a gut microbial tyrosine ammonia lyase (TAL) converts tyrosine to p-coumaric acid (pCA).

FIG. 12A-C show the creation of a competition between E. coli strains harboring hHPPD and BoTAL. FIG. 12A shows the spent media from cultures of E. coli BL21 (DE3) harboring BoTAL and hHPPD while induced with 1 mM IPTG and in the presence of 2 mM tyrosine were utilized to test pigment production. FIG. 12B shows the quantification of pyomelanin production normalized by cell density for each sample is presented in graphical format where ANOVA test was used to calculate statistical significance and p-values, n=3. The p-values for this graph indicate the significant or non-significant differences of pyomelanin produced in comparison to the pigment production in the E. coli culture harboring hHPPD. FIG. 12C shows concentrations of secreted metabolites within the spent media of synthetic communities harboring hHPPD and BoTAL as compared to individual cultures. Each grey region represents an individual sample, while the different colors in each grey region represent separate metabolites. ANOVA test was used to calculate statistical significance and p-values, n=3. The p-values show the significance between HG production in samples where hHPPD is in competition with BoTAL, compared to when hHPPD is alone.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, cell culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers to a temperature of about 15-30° C., including all integers and endpoints within the specified range. In another aspect, “room temperature” refers to a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure.

As used herein, the terms “control” or “reference” are interchangeable. A “reference” or “control” level may be a predetermined value or range, which is employed as a baseline or benchmark against which to assess a measured result. “Control” also refers to control experiments or control cells.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount” refer to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particularly in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifested.

As used herein, “tyrosine pathway intermediate” refers to any molecule that is a reactant or product in one or more of the enzymatic reactions of tyrosine metabolism in an organism. Non-limiting examples of tyrosine pathway intermediates include tyrosine, p-coumaric acid, 4-hydroxyphenylpyruvate, homogentisate, hydroxymandelate, and 4-hydroxypropionate.

Tyrosine, a versatile amino acid that undergoes diverse transformations to produce both beneficial and detrimental metabolites. The modulation of these metabolites results from direct competition among different metabolic pathways responsible for the breakdown of tyrosine, whether it be the competition between distinct microbes or the rivalry between a microbe and its host. The fight between microbes for the available tyrosine might drive potential changes to the communities present in various environments. In contrast, if a similar contest for tyrosine is presented between a gut microbial pathway and a human pathway, it can hold potential to affect human health. In this work, we present various metabolic outcomes of tyrosine within synthetic microbial communities which are prominently driven by specific enzyme activities of tyrosine breakdown pathways. Additionally, we developed a metabolic diversion of the human tyrosine breakdown pathway facilitated by a gut microbial enzyme. This approach holds promise as a novel strategy to develop potential therapeutic interventions in the future for addressing metabolic disorders like tyrosinemias (I, II, III), hawkinsinuria, and alkaptonuria, associated with the human tyrosine breakdown pathway.

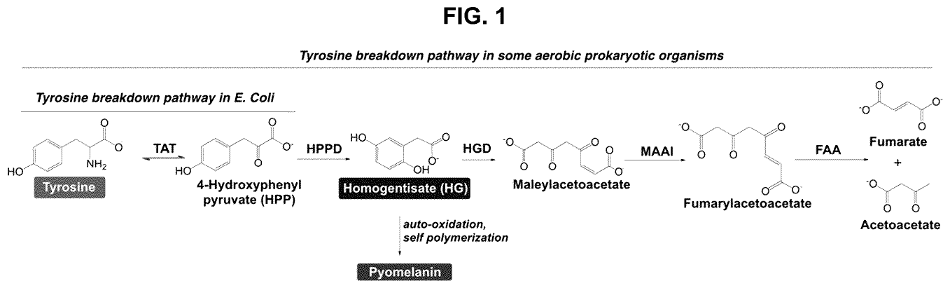

Described herein are methods for creating a bifurcation in the human tyrosine metabolism pathway that can lead to the breakdown of tyrosine without the production of harmful metabolites of the homogentisate dependent tyrosine breakdown pathway (FIG. 1) and could allow the constant utilization of tyrosine and hence may obstruct the increase in blood tyrosine levels. Utilizing synthetic communities created with E. coli strains and by simulating pathways of tyrosine breakdown that are found in gut microbes and in humans, we show evidence of pathway diversions. Our results show that these metabolic rerouting arise due to (1) the direct competition between enzymes from the members of the environmental microbial communities; and (2) the competition between a gut microbial enzyme and a human enzyme. Additionally, rather than blocking the tyrosine pathway that can increase insoluble tyrosine in the blood, utilizing a gut microbial enzyme to divert the harmful pathway and creating such a continuous clearance system for tyrosine from the blood might help with developing a better therapeutic strategy in the future.

Described herein are methods for diverting tyrosine metabolism which can be adapted to various ecosystems, such as in a synthetic microbial community. Tyrosine, a proteinogenic amino acid, undergoes diverse metabolic transformations through multiple enzymatic pathways. In aerobic organisms, tyrosine catabolism proceeds through a multi-step pathway that produces intermediates, including 4-hydroxyphenylpyruvate and homogentisate, ultimately yielding fumarate and acetoacetate. However, alternative enzymatic pathways exist that can convert tyrosine or other tyrosine pathway intermediates to different metabolites.

In one aspect, methods and compositions are described that leverage enzyme competition within synthetic microbial communities to redirect metabolic flux from one tyrosine breakdown pathway to another. When multiple enzymes capable of acting on tyrosine or another tyrosine pathway intermediate are present within the same environment, the relative activities of these enzymes determine the distribution of metabolic products. By combining microbial strains expressing different tyrosine-metabolizing enzymes, a synthetic microbial community may be constructed in which the enzymes compete for available substrate, thereby diverting tyrosine metabolism away from one pathway and toward an alternative pathway.

The methods described herein have therapeutic relevance to metabolic disorders associated with the human tyrosine breakdown pathway. In humans, the tyrosine catabolism pathway involves five enzymatic steps, and mutations or abnormal activities in enzymes of this pathway can lead to accumulation of harmful intermediates. Such accumulation is associated with metabolic disorders, including type I, type II, and type III tyrosinemias, hawkinsinuria, and alkaptonuria. Current therapeutic approaches for these disorders include administration of inhibitors that block the tyrosine breakdown pathway. However, pathway inhibition can result in elevated blood tyrosine concentrations, which may cause physiological abnormalities due to the low solubility of tyrosine.

The methods and compositions described herein provide an alternative approach in which tyrosine metabolism is diverted from the homogentisate-dependent pathway to alternative pathways that do not produce harmful intermediates. By introducing enzymes that compete with enzymes of the homogentisate pathway for tyrosine or other tyrosine pathway intermediates, the metabolic flux through the homogentisate pathway may be reduced while maintaining continuous utilization of tyrosine. This approach may address both the accumulation of harmful pathway intermediates and the elevation of blood tyrosine concentrations associated with pathway inhibition strategies.

One embodiment described herein is a method for analyzing competition for tyrosine breakdown, comprising: providing a first microbial strain expressing a first enzyme that converts a tyrosine pathway intermediate to a first metabolite; providing a second microbial strain expressing a second enzyme that converts the tyrosine pathway intermediate to a second metabolite different from the first metabolite; optionally providing a third microbial strain expressing a third enzyme that converts tyrosine to a third metabolite different from the first metabolite and the second metabolite; combining the first microbial strain, the second microbial strain, and the third microbial strain, if present, to form a synthetic microbial community; culturing the synthetic microbial community in a medium comprising tyrosine, thereby creating competition between the first enzyme, the second enzyme, and the third enzyme, if present. In one aspect, the method further comprises: measuring a quantity of the first metabolite, the second metabolite, or the third metabolite, wherein the first enzyme, the second enzyme, and the third enzyme, if present, compete for the tyrosine pathway intermediate or tyrosine, thereby diverting tyrosine metabolism from a first pathway producing the first metabolite toward a second pathway producing the second metabolite or a third pathway producing the third metabolite. In another aspect, the first microbial strain, the second microbial strain, and the third microbial strain, if present, are Escherichia coli strains. In another aspect, the Escherichia coli strains are E. coli BL21 (DE3) strains. In another aspect, the tyrosine pathway intermediate is 4-hydroxyphenylpyruvate. In another aspect, the first enzyme is a 4-hydroxyphenylpyruvate dioxygenase that converts 4-hydroxyphenylpyruvate to homogentisate. In another aspect, the 4-hydroxyphenylpyruvate dioxygenase is from Streptomyces avermitilis or Homo sapiens. In another aspect, the second enzyme is a hydroxymandelate synthase that converts 4-hydroxyphenylpyruvate to hydroxymandelate. In another aspect, the hydroxymandelate synthase is from Amycolatopsis orientalis. In another aspect, the second enzyme is a tyrosine ammonia lyase that converts tyrosine to p-coumaric acid or the third enzyme is a tyrosine ammonia lyase that converts tyrosine to p-coumaric acid. In another aspect, the tyrosine ammonia lyase is from Flavobacterium johnsoniae. In another aspect, the tyrosine ammonia lyase is from Bacteroides ovatus. In another aspect, the first microbial strain and the second microbial strain are combined at a ratio between about 1:4 and about 4:1. In another aspect, measuring a quantity of the first metabolite, the second metabolite, or the third metabolite comprises: centrifuging the medium to form a supernatant and a pellet; and measuring an absorbance of the supernatant, wherein the first metabolite is pyomelanin.

Another embodiment described herein is a composition comprising: a first recombinant Escherichia coli strain comprising an expression vector encoding a 4-hydroxyphenylpyruvate dioxygenase capable of converting 4-hydroxyphenylpyruvate (HPP) into homogentisate (HG), wherein the first recombinant Escherichia coli strain produces pyomelanin by secreting HG; a second recombinant Escherichia coli strain comprising an expression vector encoding a second enzyme that converts a tyrosine pathway intermediate to a second metabolite; and optionally a third recombinant Escherichia coli strain comprising an expression vector encoding a third enzyme that converts tyrosine to a third metabolite different from the first and second metabolites; wherein the first recombinant Escherichia coli strain produces a quantity of pyomelanin inversely proportional to an activity of the second enzyme. In one aspect, the first enzyme is a 4-hydroxyphenylpyruvate dioxygenase from Streptomyces avermitilis or a 4-hydroxyphenylpyruvate dioxygenase from Homo sapiens. In another aspect, the second enzyme is a hydroxymandelate synthase from Amycolatopsis orientalis or a tyrosine ammonia lyase from Bacteroides ovatus. In another aspect, the third enzyme is a tyrosine ammonia lyase from Flavobacterium johnsoniae. In another aspect, the first recombinant Escherichia coli strain, the second recombinant Escherichia coli strain, and the third recombinant Escherichia coli strain, if present, are E. coli BL21 (DE3) strains. In another aspect, the ratio of the first recombinant Escherichia coli strain to the second recombinant Escherichia coli strain is between about 1:4 and about 4:1.

Another embodiment described herein is a method for detecting a concentration of one or more tyrosine pathway intermediates in a sample, the method comprising: administering to the sample one or more bacterial strains comprising an expression vector encoding an enzyme selected from the group consisting of: a 4-hydroxyphenylpyruvate dioxygenase from Streptomyces avermitilis or from Homo sapiens; a hydroxymandelate synthase from Amycolatopsis orientalis; and a tyrosine ammonia lyase from Bacteroides ovatus or Flavobacterium johnsoniae; centrifuging the sample to isolate a supernatant; and measuring the absorbance of the supernatant to determine a concentration of pyomelanin. In one aspect, measuring the absorbance of the supernatant comprises analyzing the supernatant via HPLC.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Materials

All restriction enzymes, competent cells and Monarch® Plasmid Miniprep Kit were purchased from New England BioLabs (NEB). Isopropyl β-D-1-thiogalactopyranoside (IPTG) and L-tyrosine disodium salt dihydrate 98% were both purchased from Sigma Aldrich. Costar 96 well, flat, clear-bottom plate were from fisher scientific. Precision Plus protein standards ladder, in a BIO-RAD Mini-PROTEAN® TGX™ gel within a Mini-PROTEAN® Tetra Cell holding 1×Tris/Glycine/Sodium dodecyl sulfate polyacrylamide (SDS) Buffer. Competent cells of E. coli BL21(DE3)pLysS and Rosetta (DE3) were from Novagen. D/L-4-hydroxymandelic acid monohydrate and homogentisic acid were from TCI Chemicals. p-coumaric acid and M9 minimal media were purchased from Sigma Aldrich.

Pyomelanin Production in Mixed Cultures of E. coli BL21 (DE3) with pET17b-SaHPPD and E. coli BL21 (DE3) with pET17b-AoHMS

The genes for 4-hydroxyphenylpyruvate dioxygenase (SaHPPD) from Streptomyces avermitilis (SEQ ID NO: 1) and hydroxymandelate synthase (AoHMS) from Amycolatopsis orientalis (SEQ ID NO: 3) cloned in pET17b vector were received as a gift from Dr. Graham R. Moran. SaHPPD was cloned between NdeI and BamHI sites (SEQ ID NO: 11), whereas AoHMS was cloned between NdeI and XhoI sites (SEQ ID NO: 12). These plasmids were transformed into two separate E. coli strains, NEB 5α and BL21 (DE3). These cells were stored at −80° C. as glycerol stocks.

After streaking the 20% glycerol stocks of both E. coli BL21 (DE3) pET17b SaHPPD and E. coli BL21 (DE3) pET17b AoHMS on individual LB agar with 100 μg/mL ampicillin plates, they incubated overnight in 37° C. to have single colony formation. Single colonies of E. coli BL21 (DE3) containing pET17b-SaHPPD and E. coli BL21 (DE3) containing pET17b-AoHMS were used to start 5 mL starter cultures in LB with containing 100 μg/mL ampicillin. These cultures grew overnight at 37° C. with shaking at 200 rpm. Subsequently, these starter cultures were used to inoculate 50 mL cultures in LB containing 100 μg/mL ampicillin. The cultures were monitored by measuring OD₆₀₀ for growth. Once the OD₆₀₀ reached 0.6, these larger cultures were then subdivided into several 50 mL conical tubes containing 2 mM tyrosine and 1 mM IPTG in a total volume of 5 mL. 5 mL cultures of several different combinations of E. coli BL21 (DE3) containing pET17b-SaHPPD and E. coli BL21 (DE3) containing pET17b-AoHMS were created as follows: HMS:HPPD at 1:1, 3 HMS:2 HPPD, 3 HPPD:2 HMS, 4 HMS:1HPPD, and 4 HPPD:1 HMS. These test cultures were then placed at a slant on a shaker at 230 rpm and were cultivated at 37° C. for 24 h. After the 24 h of incubation, the cultures were analyzed by recording the OD 600 (600 nm) and pigmentation (405 nm) absorbance values via microplate reader. For pigmentation measurements, cultures were centrifuged and then the supernatants underwent absorbance measurements at 405 nm. There were three independent replicates (n=3) for this experiment. GraphPad Prism was used to generate a bar graph from OD₆₀₀ and 405 nm absorbance values, standard deviations and to conduct an ANOVA test to verify statistical significance. Pigmentation measurements at 405 nm were normalized to the cell density (OD) measured at 600 nm. This is to make sure that the observed pigmentation in various cultures is not due to the differences in the cell growth.

Cloning of AoHMS and SaHPPD into pET28a

SaHPPD and AoHMS were then subcloned in pET28a. SaHPPD was cloned between NdeI and BamHI sites, whereas AoHMS was cloned between NdeI and XhoI sites and transformed into E. coli NEB DH5a strains. Plasmids containing both SaHPPD and AoHMS underwent DNA sequencing at plasmidsaurus. After sequence verification, both plasmids were transformed into E. coli BL21 (DE3) strain for expression. Additionally, pET28a SaHPPD and pET28a AoHMS plasmids were transformed into another E. coli BL21 (DE3) derivative, E. coli BL21 (DE3) pLysS, where the competent cells were purchased from NEB.

Pyomelanin Production from E. coli BL21 (DE3) and E. coli BL21 (DE3) pLysS Strains Harboring pSaHPPD

E. coli BL21 (DE3) containing pET28a-SaHPPD and E. coli BL21 (DE3) pLysS containing pET28a-SaHPPD were individually grown in 5 mL LB containing 50 g/mL kanamycin. Cultures were grown within 50 ml conical tubes overnight at 37° C. at a slant with shaking at 230 rpm. These initial cultures were used to inoculate larger 50 mL subcultures in LB containing 50 μg/mL kanamycin within 125 mL Erlenmeyer flasks that grew at 37° C. with shaking at 230 rpm. Their growth was monitored by measuring culture density at OD₆₀₀ using a Costar 96 well, flat, clear-bottom plate on a microplate reader at 600 nm until they reached 0.6 optical density (OD). Once the OD₆₀₀ was reached, 5 mL of the subcultures were aliquoted into four different 50 ml conical tubes per E. coli strain containing SaHPPD which were designed for different testing conditions. The four conditions were: (1) without IPTG or tyrosine; (2) with 1 mM IPTG; (3) with 2 mM tyrosine; and (4) with both 1 mM IPTG and 2 mM tyrosine. These cultures were incubated at 37° C. with shaking at 230 rpm for 24 hours. After the 24 h incubation, 2 mL of each culture from each condition was pipetted into sterile 2 mL eppendorfs and underwent centrifugation at 1000×g for around ten minutes to collect supernatants. These supernatants were then transferred to a 96-well plate to analyze the brown pigment (pyomelanin) production within each test condition by measuring absorbance at 405 nm via a microplate reader. Separated pellets were stored in −80° C. until further use. There were three independent replicates (n=3) for this experiment. GraphPad Prism was used to generate a bar graph from OD₆₀₀ and 405 nm absorbance values, standard deviations and to conduct an ANOVA test to verify statistical significance. Pigmentation measurements at 405 nm were normalized to the cell density (OD) measured at 600 nm. This is to make sure that the observed pigmentation in various cultures is not due to the differences in the cell growth.

Protein Expression Analysis Via SDS-PAGE from E. coli BL21 (DE3) and E. coli BL21 (DE3) pLysS Strains Harboring pSaHPPD

The pellets collected from previous pigmentation cultures were thawed on ice and resuspended in 1.5 mL of sterile 1×M9 minimal media (sterile buffer). The optical densities of these resuspended samples were normalized and then 15 μL of each sample was mixed with 15 L of Laemmli sample buffer containing dye and beta-mercaptoethanol (BME). The samples were placed in boiling water bath for ten minutes and then centrifuged for 5 min at 1000×g and finally loaded alongside the Precision Plus protein standards ladder, in a BIO-RAD Mini-protean® TGX™ gel for SDS-PAGE analysis. After the run the gel was stained with QC colloidal coomassie stain and was imaged with a BIO-RAD gel imager to confirm the protein band sizes.

Pyomelanin Production in Mixed Cultures of E. coli BL21 (DE3) pLysS Containing pET28a-SaHPPD, E. coli BL21 (DE3) pLysS pET28a-AoHMS, and E. coli Rosetta (DE3) Containing pET28a-FjTAL

The gene for tyrosine ammonia lyase (FjTAL) (Gene ID: 644740509) from Flavobacterium johnsoniae (SEQ ID NO: 5) was PCR amplified from the F. johnsoniae genome and cloned into pET28a vector between NdeI and XhoI restriction sites. This newly made pET28a-FjTAL was then transformed into E. coli Rosetta (DE3). pET28a-FjTAL was transformed to Rosetta to assure proper expression of FjTAL due to its ability to harbor tRNAs for expression of proteins that are non-native to E. coli or not codon optimized. Additionally, due to the requirement of chloramphenicol for E. coli Rosetta (DE3), we subcloned both SaHPPD and AoHMS in the E. coli BL21 (DE3) pLysS strain that is also resistant to chloramphenicol. All three of these strains were plated on LB agar plates containing 50 μg/mL kanamycin and 25 μg/mL chloramphenicol to account for resistance against chloramphenicol and kanamycin resistance coming from pET28a vector. Plasmids from all three strains were sequenced through Plasmidsaurus, then 20% glycerol stocks were created and stored in −80° C. until further use.

Competition assays were carried out similar to AoHMS and SaHPPD competition assays as mentioned above. Single colonies from each of the three strains mentioned above were used to create three 5 mL LB starter cultures containing 50 μg/mL kanamycin and 25 μg/mL chloramphenicol. These cultures grew overnight at 37° C. with shaking at 230 rpm. Subsequently, these starter cultures were used to inoculate 50 mL cultures in LB containing 50 μg/mL kanamycin and 25 μg/mL chloramphenicol. The cultures were monitored by measuring OD₆₀₀ for growth. Once the OD₆₀₀ reached 0.6, these larger cultures were then subdivided into several 50 mL conical tubes containing 2 mM tyrosine and 1 mM IPTG in a total volume of 5 mL. 5 mL cultures of several different combinations of E. coli BL21 (DE3) pLysS containing pET28a-SaHPPD, E. coli BL21 (DE3) pLysS containing pET28a-AoHMS, and E. coli Rosetta (DE3) containing pET28a-FjTAL were created as follows: only TAL, only HMS, only HPPD, HMS:HPPD at 1:1, TAL:HMS at 1:1, TAL:HPPD at 1:1, and HPPD:HMS:TAL at 1:1:1. These test cultures were then placed at a slant on a shaker at 230 rpm and were cultivated at 37° C. for 24 h. After the 24 h of incubation, the cultures were analyzed by recording the OD₆₀₀ (600 nm) and pigmentation (405 nm) absorbance values via microplate reader. For pigmentation measurements, cultures were centrifuged and then the supernatants underwent absorbance measurements at 405 nm. There were three independent replicates (n=3) for this experiment. GraphPad Prism was used to generate a bar graph from OD₆₀₀ and 405 nm absorbance values, standard deviations and to conduct an ANOVA test to verify statistical significance. Pigmentation measurements at 405 nm were normalized to the cell density (OD) measured at 600 nm. This is to make sure that the observed pigmentation in various cultures is not due to the differences in the cell growth.

Pyomelanin Production in Mixed Cultures of E. coli BL21 (DE3) Containing pET28a-BoTAL and E. coli BL21 (DE3) containing pET28a-hHPPD

Genes for tyrosine ammonia lyase (BoTAL) (Gene ID: 29455038) from Bacteroides ovatus (BoTAL) (SEQ ID NO: 9) and human 4-hydroxyphenylpyruvate dioxygenase (hHPPD) (Gene ID: 3242) (SEQ ID NO: 7), synthesized and codon optimized for E. coli expression, were received from GeneWiz®. These genes were then cloned into the pET28a vector, where hHPPD was cloned between NcoI and BamHI (SEQ ID NO: 13) and BoTAL was cloned between NdeI and BamHI sites (SEQ ID NO: 14). Both vectors pET28a-hHPPD and pET28a-BoTAL were then transformed into E. coli NEB DH5a and E. coli BL21 (DE3) competent cells and glycerol stocks were stored in −80° C. until further use.

The same procedures as previously stated above were used to analyze pyomelanin production with E. coli BL21 (DE3) containing pET28a-hHPPD and E. coli BL21 (DE3) containing pET28a-BoTAL mixed culture competition assays. The combinations for mixed cultures of hHPPD and BoTAL were: only TAL, only HPPD, and HPPD:TAL at 1:1, 3 TAL: 2 HPPD, 3 HPPD: 2 TAL, 4 TAL: 1 HPPD, 4 HPPD: 1 TAL. These test cultures were then placed at a slant on a shaker at 230 rpm and were cultivated at 37° C. for 24 h. After the 24 h of incubation, the cultures were analyzed by recording the OD₆₀₀ (600 nm) and pigmentation (405 nm) absorbance values via a microplate reader. For pigmentation measurements, cultures were centrifuged and then the supernatants underwent absorbance measurements at 405 nm. There were three independent replicates (n=3) for this experiment. GraphPad Prism was used to generate a bar graph from OD₆₀₀ and 405 nm absorbance values, standard deviations and to conduct an ANOVA test to verify statistical significance. Pigmentation measurements at 405 nm were normalized to the cell density (OD) measured at 600 nm. This is to make sure that the observed pigmentation in various cultures is not due to the differences in the cell growth.

Table 1 shows the nucleotide and amino acid sequences for each of the Sa HPPD, Ao HMS, Fj TAL, hHPPD, and Bo TAL enzymes.

TABLE 1
Nucleotide and Amino Acid Sequences

Sa HPPD Nucleotide Sequence SEQ ID NO: 1
atgacgcagaccacccaccacactcccgacaccgcccggcaggccgaccccttcccggtgaagggaatggacgcgg
tcgtcttcgccgtaggcaacgccaagcaggccgcgcactactactccaccgccttcggcatgcagcttgtggcgta
ctccggaccggagaacggcagccgcgagaccgcttcgtacgtcctcaccaacggctcggcacgcttcgtcctcacc
tccgtcatcaagcccgccaccccctggggccacttcctcgccgaccatgtggccgagcacggcgacggcgtcgtcg
acctcgccatcgaggtcccggacgcccgcgccgcccacgcgtacgcgatcgagcacggcgcccgctcggtcgccga
gccgtacgagctgaaggacgagcacggcacggtcgtcctcgccgcgatcgccacctacggcaagacccgccacacc
ctcgtcgaccggaccggctacgacggcccctacctccccggctacgtggccgccgccccgatcgtcgaaccgcccg
cccaccgcaccttccaggccatcgaccactgcgtcggcaacgtcgagctcggccggatgaacgaatgggtcggctt
ctacaacaaggtcatgggcttcacgaacatgaaggagttcgtgggcgacgacatcgcgaccgagtactcggcgctg
atgtcgaaggtcgtggccgacggcacgctcaaggtcaagttcccgatcaacgagcccgccctcgccaagaagaagt
cccagatcgacgagtacctggagttctacggcggcgcgggcgtccagcacatcgcgctgaacacgggtgacatcgt
cgagacggtacgcacgatgcgcgccgccggcgtccagttcctggacacgcccgactcgtactacgacaccctcggg
gagtgggtgggcgacacccgcgtccccgtcgacaccctgcgcgagctgaagatcctcgcggaccgcgacgaggacg
gctatctgctccagatcttcaccaagccggtccaggaccgcccgacggtcttcttcgagatcatcgaacgccacgg
ctcgatgggattcggcaagggcaacttcaaggccctgttcgaggcgatcgagcgggagcaggagaagcggggcaac
ctg
Sa HPPD Amino Acid Sequence SEQ ID NO: 2
MTQTTHHTPDTARQADPFPVKGMDAVVFAVGNAKQAAHYYSTAFGMQLVAYSGPENGSRETASYVLTNGSARFVLT
SVIKPATPWGHFLADHVAEHGDGVVDLAIEVPDARAAHAYAIEHGARSVAEPYELKDEHGTVVLAAIATYGKTRHT
LVDRTGYDGPYLPGYVAAAPIVEPPAHRTFQAIDHCVGNVELGRMNEWVGFYNKVMGFTNMKEFVGDDIATEYSAL
MSKVVADGTLKVKFPINEPALAKKKSQIDEYLEFYGGAGVQHIALNTGDIVETVRTMRAAGVQFLDTPDSYYDTLG
EWVGDTRVPVDTLRELKILADRDEDGYLLQIFTKPVQDRPTVFFEIIERHGSMGFGKGNFKALFEAIEREQEKRGN
L
Ao HMS Nucleotide Sequence SEQ ID NO: 3
atgcagaatttcgagatcgactacgtagaaatgtatgtggaaaatcttgaggtggctgcgttcagctgggtggaca
agtacgcgttcgccgtcgccggtaccagccggtcggcggaccaccggagcatcgcgctacggcaggggcaggtgac
gctggtcctcaccgagcccacgtcggatcgtcacccggcggcggcctacctccagacgcacggcgacggtgtcgcc
gacatcgcgatggccacttcggacgtcgccgccgcttacgaggccgcggtgcgggcgggggcggaagccgtccgcg
cgccggggcagcactcggaggccgcggtcacgacggccaccatcggtggtttcggcgatgtcgtgcacaccctgat
tcagcgcgacggaacatccgccgagctgcccccgggcttcaccggctcgatggacgtcaccaaccacggcaaggga
gatgtcgacctgctcggcatcgaccacttcgcgatctgcctgaacgccggcgacctcggccccacggtggagtact
acgagcgggcactgggtttccggcagatcttcgacgagcacatcgtggtcggtgcgcaggcgatgaactccaccgt
cgtgcagagtgcgtccggagcggtcaccctcaccctgatcgagcccgaccggaacgccgaccccggccagatcgac
gagttcctcaaggatcaccaaggggcaggcgttcagcacatcgccttcaacagcaacgacgcggtccgtgcggtca
aggcgctgtccgagcgcggggtggagttcctcaagacgccgggggcctactacgacctgctcggtgagcggatcac
gctgcagacgcactcgctggacgatctgcgggcgacgaacgtgctcgccgatgaggaccacggcggccagctgttc
cagatcttcaccgcgtccactcatccgcgtcacacgattttcttcgaggtcatcgagcggcagggcgcgggcacct
tcggcagctccaacatcaaggccctgtacgaggccgttgagctggaacgcaccgggcagagcgagttcggcgccgc
tcggcgat
Ao HMS Amino Acid Sequence SEQ ID NO: 4
MQNFEIDYVEMYVENLEVAAFSWVDKYAFAVAGTSRSADHRSIALRQGQVTLVLTEPTSDRHPAAAYLQTHGDGVA
DIAMATSDVAAAYEAAVRAGAEAVRAPGQHSEAAVTTATIGGFGDVVHTLIQRDGTSAELPPGFTGSMDVTNHGKG
DVDLLGIDHFAICLNAGDLGPTVEYYERALGFRQIFDEHIVVGAQAMNSTVVQSASGAVTLTLIEPDRNADPGQID
EFLKDHQGAGVQHIAFNSNDAVRAVKALSERGVEFLKTPGAYYDLLGERITLQTHSLDDLRATNVLADEDHGGQLF
QIFTASTHPRHTIFFEVIERQGAGTFGSSNIKALYEAVELERTGQSEFGAARR
Fj TAL Nucleotide Sequence SEQ ID NO: 5
atgaatacaattaacgaatatttaagtttagaagaatttgaggccataatctttggaaatcaaaaagttacaatta
gcgacgttgtcgtaaaccgagttaatgaaagtttcaatttcttaaaagaattctctgggaataaagtaatttacgg
cgtaaacactggttttggaccaatggcgcagtatagaattaaagagtctgatcaaattcagcttcaatataattta
attagaagccactcttcgggaaccggaaagccattaagcccggtttgtgctaaagcagcaattttagctcgtttaa
acactctttcattaggaaattcaggcgttcacccgtcagtaatcaacctaatgtctgagcttatcaataaagatat
tacccctttaatcttcgaacacggaggcgttggtgccagcggtgacttagtacagctgtcacatttagctttagtg
ttaattggcgaaggtgaagttttttataaaggtgaaagaagaccaactcctgaagtttttgaaattgaaggtttaa
aaccaattcaggtagaaattagagaaggtctggcattaattaacggaacttcagtaatgactggaattggagttgt
aaatgtttatcatgctaaaaaattattagactggtcattaaaatcttcttgtgcaattaacgaattggttcaggct
tatgacgatcatttctcagcagaactaaaccagactaaacgtcataaaggacagcaggaaattgcgttgaaaatgc
gtcaaaatctttcagacagtactttaatccgtaaaagagaagatcatttatattctggtgaaaataccgaagaaat
cttcaaagaaaaagttcaggaatattattcattaagatgcgttcctcaaattttaggaccggttttagaaactatt
aataatgtagcttctattctggaagacgaatttaactcggcaaacgataacccaattatcgatgtaaaaaaccagc
atgtttaccacggcggaaatttccacggagattatatttcgcttgaaatggataaactaaaaattgttattaccaa
attaacaatgctggcagaacgtcagttgaactatttattaaattcaaaaatcaacgaattacttcctccgtttgta
aacttaggaacattaggatttaatttcggaatgcagggtgttcagtttacagcaacatctacaacggcagaaagtc
aaatgctgtcgaacccaatgtatgttcacagtattccaaacaacaacgacaatcaggatatcgtaagtatgggaac
taactcggcagtaattacctcaaaagtaatcgaaaatgcttttgaagttctggctatcgaaatgattactatcgtt
caggcaattgattatttaggacaaaaagacaaaatttcatctgtttctaaaaaatggtacgatgaaattagaaata
taattccaacatttaaggaagatcaggtaatgtatcctttcgttcaaaaagtaaaagatcatttgatcaacaatta
a
Fj TAL Amino Acid Sequence SEQ ID NO: 6
MNTINEYLSLEEFEAIIFGNQKVTISDVVVNRVNESFNFLKEFSGNKVIYGVNTGFGPMAQYRIKESDQIQLQYNL
IRSHSSGTGKPLSPVCAKAAILARLNTLSLGNSGVHPSVINLMSELINKDITPLIFEHGGVGASGDLVQLSHLALV
LIGEGEVFYKGERRPTPEVFEIEGLKPIQVEIREGLALINGTSVMTGIGVVNVYHAKKLLDWSLKSSCAINELVQA
YDDHFSAELNQTKRHKGQQEIALKMRQNLSDSTLIRKREDHLYSGENTEEIFKEKVQEYYSLRCVPQILGPVLETI
NNVASILEDEFNSANDNPIIDVKNQHVYHGGNFHGDYISLEMDKLKIVITKLTMLAERQLNYLLNSKINELLPPFV
NLGTLGFNFGMQGVQFTATSTTAESQMLSNPMYVHSIPNNNDNQDIVSMGTNSAVITSKVIENAFEVLAIEMITIV
QAIDYLGQKDKISSVSKKWYDEIRNIIPTFKEDQVMYPFVQKVKDHLINN
hHPPD Nucleotide Sequence SEQ ID NO: 7
atggccatgaccacctatagcgataaaggcgcgaaaccggaacgcggccgctttctgcattttcatagcgtgacct
tttgggtgggcaacgcgaaacaagcggcgagcttttattgcagcaaaatgggctttgaaccgctggcgtatcgcgg
actggaaaccggcagccgcgaagtggtgagccatgtgattaaacaaggcaaaattgtgtttgtgctgagcagcgcg
ctgaacccgtggaacaaagaaatgggcgatcatctggtgaaacatggcgatggcgtgaaagatattgcgtttgaag
tggaagattgcgattatattgtgcagaaagcgcgtgaacgcggcgcgaaaattatgcgcgaaccgtgggtggaaca
agataaatttggcaaagtgaaatttgcggtgctgcagacctatggcgataccacccataccctggtggaaaaaatg
aactatattggtcagtttctgccgggctatgaagcgccggcgtttatggacccgctgctgccgaaactgccgaaat
gcagcctggaaatgattgatcatattgtgggcaatcagccggatcaagaaatggtgagcgcgagcgaatggtatct
gaaaaacctgcagtttcatcgcttttggagcgtggatgatacccaagtgcataccgaatatagcagcctgcgcagc
attgtggtggcgaactatgaagaaagcattaaaatgccgattaacgaaccggcgccgggcaaaaaaaaaagtcaga
ttcaagaatatgtggattataacggcggcgcgggcgtgcagcatattgcgctgaaaaccgaagatattattaccgc
gattcgccatctgagagaacgcggcctggaatttctgagcgtgccgagcacctattataaacagctgcgcgaaaaa
ctgaaaaccgcgaaaattaaagtgaaagaaaacattgatgcgctggaagaactgaaaattctggtggattatgatg
aaaaaggctatctgctgcagatttttaccaaaccggtgcaagatcgcccgaccctgtttctggaagtgattcagcg
ccataaccatcaaggctttggcgcgggcaactttaacagcctgtttaaagcgtttgaagaagaacagaacctgcgc
ggcaacctgaccaacatggaaaccaacggcgtggtgccgggcatgtaa
hHPPD Amino Acid Sequence SEQ ID NO: 8
MTTYSDKGAKPERGRFLHFHSVTFWVGNAKQAASFYCSKMGFEPLAYRGLETGSREVVSHVIKQGKIVFVLSSALN
PWNKEMGDHLVKHGDGVKDIAFEVEDCDYIVQKARERGAKIMREPWVEQDKFGKVKFAVLQTYGDTTHTLVEKMNY
IGQFLPGYEAPAFMDPLLPKLPKCSLEMIDHIVGNQPDQEMVSASEWYLKNLQFHRFWSVDDTQVHTEYSSLRSIV
VANYEESIKMPINEPAPGKKKSQIQEYVDYNGGAGVQHIALKTEDIITAIRHLRERGLEFLSVPSTYYKQLREKLK
TAKIKVKENIDALEELKILVDYDEKGYLLQIFTKPVQDRPTLFLEVIQRHNHQGFGAGNENSLFKAFEEEQNLRGN
LTNMETNGVVPGM
Bo TAL Nucleotide Sequence SEQ ID NO: 9
atgattgcggataaaagcattaacctggataccctgcataaagtgctgtttgataacgaaaaactgaaactgagcg
aagaatgcattcgcaaagtggaagaaagctttgattttctgcagagctttagcagcgataaaattatttatggcat
taacaccggctttggcccgatggcgcagtatcgcattgaagatcagagcctgattgatctgcagtataacattatt
cgcagccatagcaccggcgcgggcaaaccgctgccggaactgtatgtgaaagcggcgatgattgcgcgcctgtata
cctttctgcaaggcaaaagcggcgtgcatctggaactggtgagcctgctgtgcgaatttattaaccgcggcattta
tccgtttattccggaacatggcagcgtgggcgcgagcggcgatctggtgcagctggcgcatattgcgctgaccctg
attggcgaaggcgaagtgttttatcaaggcaaactgtgcaacgcggcgaccgtgctgcaagaaaacggcctgaaac
cgtttagcatgcgcattcgcgaaggcctgagcgtgaccaacggcacgagcgtgatgaccggcattggcattgtgaa
cctgatttatgcgaaaaaactgctgcgctggagcgtggcggcgagcgtgatgatgaacgaaattgcagcgtcatac
gatgattttatggctcaagcgttgaacgaagcgaaacatcataaaggtcagcaagaaattgcggcgatgatgcgcg
aatgggtggcgggcagcaaaagcgtgctgcagcgcgaaaacgaactgtataaccaagtgcataaagaaaaaatttt
tgaacataaagtgcagccgtattatagcctgcgctgcgtgccgcagattctgggcccgatttatgatgaactggaa
aacgcggaagaagtgctgattaacgaaattaacagcgcgtgcgataacccgattgttgatccggatacgcagaaca
tttatcatggcggcaactttcatggcgattatattagctttgaaatggataaactgaaaattgcggtgaccaaact
gaccatgctgtgcgaacgtcagattaactatctgtttcatgatcgcattaacggcattctgccgccgtttgtgaac
ctgggcgtgctgggcctgaactatggcctgcaagcgagtcagtttaccgcgacgagcaccaccgcggaatgtcaga
ccctgagcaacccgatgtatgtgcatagcattccgaacaacaacgataaccaagatattgtgagcatgggcaccaa
cagcgcgctgctggcgaaaaccgtgattgaaaacagctatcaagtgatggcgattcagtttatgggcatggcgcaa
gcgatagattacctgaaaatccaagatcgcctgagcagcaaaagccgccaagtgtatgaagaaattcgcagctttt
ttccggtgtttaccaacgataccccgaaatataaagaaattgaaatgatgattgattacctgaagaaggaggataa
a
Bo TAL Amino Acid Sequence SEQ ID NO: 10
MIADKSINLDTLHKVLFDNEKLKLSEECIRKVEESFDFLQSFSSDKIIYGINTGFGPMAQYRIEDQSLIDLQYNII
RSHSTGAGKPLPELYVKAAMIARLYTFLQGKSGVHLELVSLLCEFINRGIYPFIPEHGSVGASGDLVQLAHIALTL
IGEGEVFYQGKLCNAATVLQENGLKPFSMRIREGLSVINGTSVMTGIGIVNLIYAKKLLRWSVAASVMMNEIAASY
DDFMAQALNEAKHHKGQQEIAAMMREWVAGSKSVLQRENELYNQVHKEKIFEHKVQPYYSLRCVPQILGPIYDELE
NAEEVLINEINSACDNPIVDPDTQNIYHGGNFHGDYISFEMDKLKIAVTKLTMLCERQINYLFHDRINGILPPFVN
LGVLGLNYGLQASQFTATSTTAECQTLSNPMYVHSIPNNNDNQDIVSMGTNSALLAKTVIENSYQVMAIQFMGMAQ
AIDYLKIQDRLSSKSRQVYEEIRSFFPVFTNDTPKYKEIEMMIDYLKKEDK

Table 2 shows the enzymes, vectors, SEQ ID NOs, and E. coli strains tested for each of the clones.

TABLE 2
Enzymes, Vectors, and E. coli Strains

| | Vector(s) | SEQ ID | |
|---|---|---|---|
| Enzyme | Tested | NO | E. coli Strain(s) Tested |
| 4-hydroxyphenylpyruvate | pET17b | 11 | E. coli BL21(DE3) |
| dioxygenase from Streptomyces | pET26b | — | E. coli BL21(DE3)pLysS |
| avermitilis (Sa HPPD) | pET28a | — | E. coli BL21(DE3) |
| 4-hydroxymandelate synthase from | pET17b | — | E. coli BL21(DE3)pLysS |
| Amycolatopsis orientalis (Ao HMS) | pET28a | — | E. coli BL21(DE3)pLysS |
| Tyrosine ammonia lyase from | pET28a | 12 | E. coli Rosetta BL21 |
| Flavobacterium johnsoniae (Fj TAL) | | | |
| 4-hydroxyphenylpyruvate from | pET28a | 13 | E. coli BL21(DE3) |
| Homo sapiens (hHPPD) | | | |
| Tyrosine ammonia lyase from | pET28a | 14 | E. coli BL21(DE3) |
| Bacteroides ovatus (Bo TAL) | | | |

HPLC of Secreted Tyrosine Metabolites from Various E. coli Strains in Mixed Competition Assay Cultures

For detection and measurement of secreted tyrosine metabolites, HPLC analysis of spent media from mixed cultures was carried out. Previously described competition assays of mixed cultures were carried out as mentioned above but instead of using LB media, the assays were conducted in M9 minimal media. Induced cultures containing 2 mM tyrosine were incubated for 48 h at 37° C. with shaking at 230 rpm. After 48 h of incubation, cells were harvested by centrifugation for all cultures and the spent media (supernatants) were collected. These supernatants (spent media) were then passed through 0.2 μm PES syringe filters to remove any cell debris and then through 10 kDa MWCO filters by centrifugation at 1000×g to remove any small peptides and proteins. The flow through from these samples were then analyzed by HPLC as a final volume of 100 μL. Each sample contained 25 UM of Thiamine HCl as an internal standard.

Separation was carried out using a Synergi 4 μm Hydro-RP 80 Å 250×4.6 mm column on Waters e2695 separation module with a diode array detector via two separate methods to analyze peaks representing tyrosine, hydroxymandelic acid (HMA), homogentisate (HG), and p-coumaric acid (pCA). For isocratic method, samples were separated under isocratic conditions with a mobile phase consisting of 20 mM sodium phosphate, pH 2 and 0.5% acetonitrile at 1 mL/min. For gradient method, mobile phase consisting of methanol-water with 0.1% formic acid (Buffer A, methanol with 0.1% formic acid and Buffer B, water with 0.1% formic acid). Briefly the method consisted of 5:95 (A:B) for 0-5 min, 5:95 to 20:80 from 5-14 min, 20:80 to 80:20 from 14-18 min, 80:20 from 18-25 min. After 25 min, column was brought back to the initial conditions before loading the new sample. Samples were monitored using DAD and all metabolites could be visualized at 276 nm.

The peak areas from chromatograms of various samples were analyzed using the chromatography data software, Empower. Concentration of metabolites were calculated from peak areas by standard curves generated for tyrosine, HMA, HG, and pCA. Finally, the values were entered into GraphPad Prism to generate a plot showing the relationship between concentrations within different mixed samples. GraphPad Prism was utilized to generate bar graphs, to compute standard deviation, and to perform ANOVA testing to provide statistical analysis of data and provide p-values.

### Pyomelanin Serves as the Indicator for Secreted Homogentisate

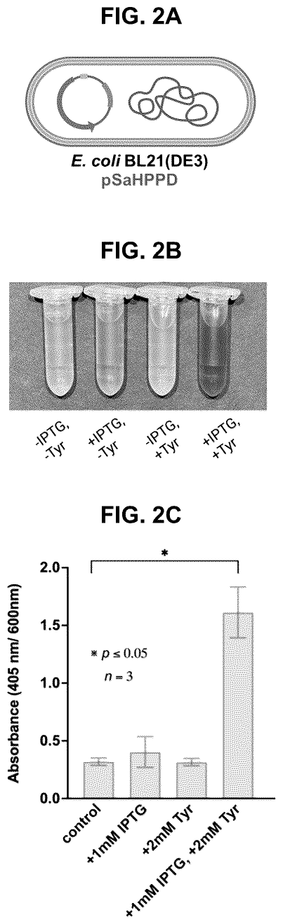

To test if a metabolic diversion could be achieved in microbial communities, we employed a phenotypic assay. An E. coli strain harboring the enzyme HPPD is able to produce a brown pigment when grown in LB media. E. coli is an excellent assay system here because it only has the first enzyme of the tyrosine breakdown pathway, known as tyrosine aminotransferase (TAT) that converts tyrosine to 4-hydroxyphenylpyruvate (4-HPP) (FIG. 1). In addition, E. coli cells are able to transport tyrosine and its metabolites across the cell. Here, we utilized an E. coli strain harboring an enzyme 4-hydroxyphenylpyruvate dioxygenase (HPPD) (FIG. 2). HPPD catalyzes the second step of the tyrosine catabolism pathway (FIG. 1). When this strain is given tyrosine, it first gets converted to 4-HPP and then to homogentisate (HG) due to the HPPD that is provided within the plasmid. Homogentisate can be secreted outside the cell, where it gets oxidized and self-polymerized to produce a brown pigment called pyomelanin. This pigment can be measured at 405 nm. We tested E. coli BL21 (DE3) for the pigment production due to the expression of the enzyme 4-hydroxyphenylpyruvate dioxygenase (HPPD) from an environmental microbe (FIG. 2).

FIG. 2 depicts pigment production due the presence of HPPD in E. coli BL21 (DE3) cells. We observed the most pigment when these cultures were induced and had an additional source of the substrate tyrosine (FIG. 2, +IPTG, +Tyr). There was some pigment production when cultures were induced without the addition of the substrate, and we believe this is due to the additional minimal source of tyrosine found in the rich media (LB) used (FIG. 2, +IPTG). FIG. 2 graph represents the quantification of the pigmentation in above mentioned cultures. Although, we didn't see any difference in the growth rates of these cultures, we normalized the absorbance coming from the pigment via OD₆₀₀ to eliminate any small differences in the number of cells that produce the pigment. There is a significant amount of pigment production in cultures that received both tyrosine and IPTG, specially for BL21 (DE3) strain (FIG. 2 graph).

Pigmentation is Proportional to Ratios of E. coli Strains Harboring Different Enzymes for Tyrosine Metabolism

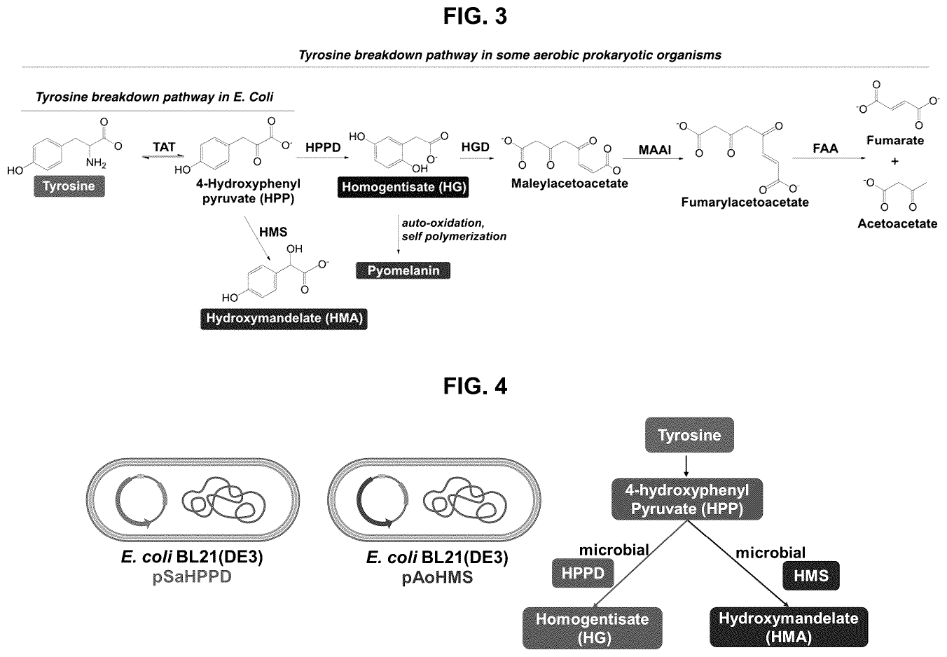

To understand the allocation of tyrosine as a resource in complex microbial communities, we started with two E. coli strains differing only by the tyrosine metabolizing enzyme present on a plasmid. For this, we chose 4-hydroxyphenylpyruvate dioxygenase (SaHPPD) from Streptomyces avermitilis and hydroxymandelate synthase (AoHMS) from Amycolatopsis orientalis (FIG. 3 and FIG. 4). Both S. avermitilis and A. orientalis are environmental microbes and have the ability to produce a variety of natural products. For example, S. avermitilis produces avermectins which are utilized as antiparasitic, insecticide and antihelminitic drugs. Whereas vancomycin, a peptide antibiotic was first isolated from A. orientalis. A not proteinogenic amino acid 4-hydroxyphenylglycine (pHPG) is needed for the production of vancomycin and hydroxymandelate (HMA), the product of the reaction catalyzed by AoHMS is the precursor for pHPG. In contrast, S. avermitilis produces multiple pigments like melanin that are produced from tyrosine. To understand the competition in utilizing tyrosine within the community of such environmental microbes, we created a synthetic community of E. coli strains harboring and differing only in the presence of tyrosine metabolism enzymes (FIG. 3 and FIG. 4).

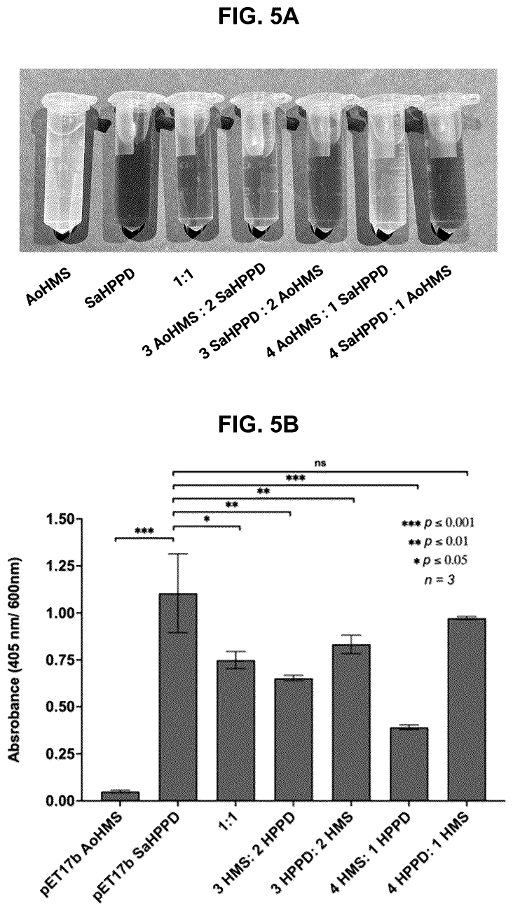

The competition between SaHPPD and AoHMS proceeds via an intermediate 4-HPP (FIG. 3 and FIG. 4). When E. coli BL21 (DE3) cells harboring either SaHPPD or AoHMS were cultivated individually or as mixtures of different ratios, the competition between pathways was visible through the change in the pyomelanin production (FIG. 5). FIG. 5 shows the pigmentation in spent media of various cultures of E. coli BL21 (DE3) cells harboring either SaHPPD or AoHMS or mixtures of the two at various ratios. The product of AoHMS is HMA that does not produce pyomelanin hence there was no pigmentation in the AoHMS culture. In contrast, homogentisate (HG), the product of SaHPPD, can produce pyomelanin hence a dark brown pigmentation was seen in the spent media of SaHPPD culture. Interestingly, when the cultures of AoHMS and SaHPPD were mixed at various ratios and then allowed to grow with induction and addition of tyrosine, the resulting cultures showed pigmentation levels that correlated very well with the initial ratios of two mixed E. coli strains, where higher amount of added E. coli cells with SaHPPD showed higher pigmentation (FIG. 5). As expected, the highest pigmentation is seen with SaHPPD culture followed by 4 SaHPPD:1 AoHMS, 3 SaHPPD:2 AoHMS, 2 SaHPPD:3 AoHMS, and then 1 SaHPPD:4 AoHMS. Culture ratio of 1 SaHPPD:1 AoHMS, showed pigmentation that was between the pigmentation level of 3 SaHPPD:2 AoHMS and 2 SaHPPD:3 AoHMS. This result showed that the direct competition between two enzymatic activities was being translated in the pigmentation differences which are ultimately driven due to changes in the metabolic output of this synthetic community.

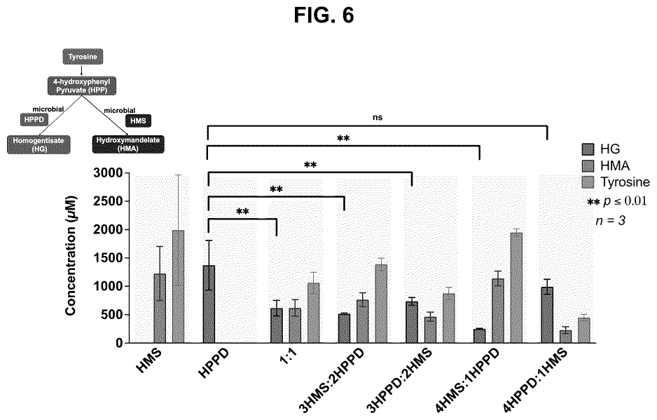

To further understand this community dynamics, we carried out HPLC analysis to detect and measure secreted metabolites from the spent media. From the spent media of various cultures, HG, HMA and tyrosine were separated and measured. FIG. 6 shows the quantification of expected metabolites from the individual and mixed culture of E. coli BL21 (DE3) SaHPPD and E. coli BL21 (DE3) AoHMS. When E. coli BL21 (DE3) SaHPPD was grown separately, we found 1370±440 UM of HG, which decreased gradually to 248±10 μM as the ratio of SaHPPD to AoHMS changed from 4:1 to 1:4 in the mixed cultures (Table 3). On the other hand, the concentrations of HMA increased concurrently with decrease in HG. Additionally, the culture ratio of 1 SaHPPD:1 AoHMS, showed pigmentation that was between the pigmentation level of 3 SaHPPD:2 AoHMS and 2 SaHPPD:3 AoHMS, and we found equimolar concentration of HG and HMA in the spent media of this 1:1 mixed culture. It is only possible to observe such results where ratios of metabolites are changing based on the changes in the abundance of enzymes, if the kinetic parameters of these enzymes are not vastly different which is the case here for HPPD and HMS.

TABLE 3
Concentration of Metabolites Measured in individual and Mixed Cultures
of E. coli BL21(DE3) SaHPPD and E. coli BL21(DE3) AoHMS

| | Homgentisate (HG) | Hydroxymandelate | |
|---|---|---|---|
| Culture | (μM) | (HMA) (μM) | Tyrosine (μM) |
| HMS | N/A | 1120 ± 480 | 1989 ± 977 |
| HPPD | 1369 ± 437 | N/A | N/A |
| 4 HPPD:1 HMS | 988 ± 133 | 225 ± 29 | 1058 ± 188 |
| 3 HPPD:3 HMS | 732 ± 68 | 463 ± 27 | 1384 ± 110 |
| 2 HPPD:3 HMS | 516 ± 11 | 762 ± 56 | 872 ± 107 |
| 1 HPPD:4 HMS | 248 ± 10 | 1136 ± 124 | 1946 ± 64 |
| 1 HPPD:1 HMS | 614 ± 137 | 617 ± 46 | 448 ± 57 |

Concentration of Metabolites Measured in Individual and Mixed Cultures of E. coli BL21 (DE3) SaHPPD and E. coli BL21 (DE3) AoHMS

In contrast, if one of the enzymes was very fast, meaning it has a large Vmax, or had significantly high affinity for the substrate, meaning a low Kₘ, then that would result in the predominance of the product of that enzyme even within the mixed cultures. In such a simple synthetic community, the competition for the resource, which is tyrosine, is thus driven directly by the competing enzyme activities. This shows that it is crucial to understand metabolic pathway diversions and kinetic parameters of enzymes involved in those pathways and such simplified synthetic microbial communities provide a unique strategy to predict metabolic outcomes. Interestingly, we observed large variations in the metabolite concentrations in pure cultures compared to mixed cultures (FIG. 6). Additionally, cultures with AoHMS showed a significant amount of tyrosine leftover or excess production in the spent media. Specifically in the case of AoHMS cultures, we saw around 2 mM tyrosine in the spent media which gradually decreased in mixed cultures with the correlating gradual increase in added HPPD. Whereas in pure SaHPPD cultures, we did not detect any tyrosine in the spent media. We think that there might be competition between AoHMS and an E. coli tyrosine aminotransferase enzyme (EcTAT) (FIG. 1). EcTAT is the only tyrosine catabolism enzyme present in E. coli cells which can catalyze a reversible conversion of tyrosine to 4-HPP and vice versa. If EcTAT is faster than AoHMS, then it might be catalyzing the conversion of 4-HPP to tyrosine faster than the conversion of 4-HPP to HMA by AoHMS and this might be resulting in the increased concentration of tyrosine in cultures that have AoHMS enzyme.

Competition for Tyrosine within a Synthetic Community of E. coli Strains is Driven by the Activities of Tyrosine Metabolism Enzymes

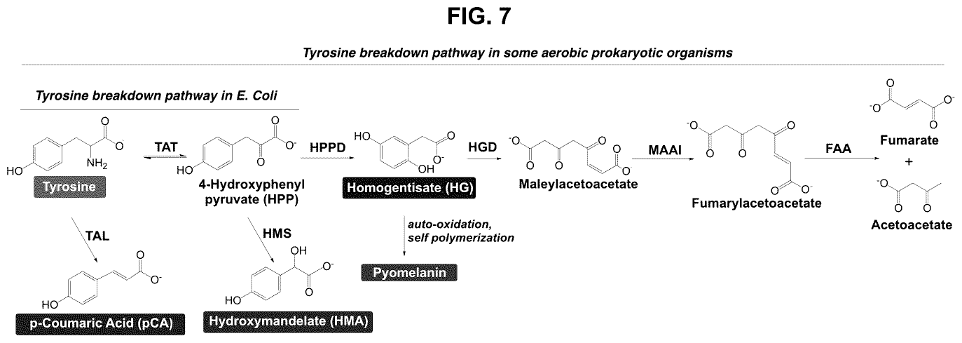

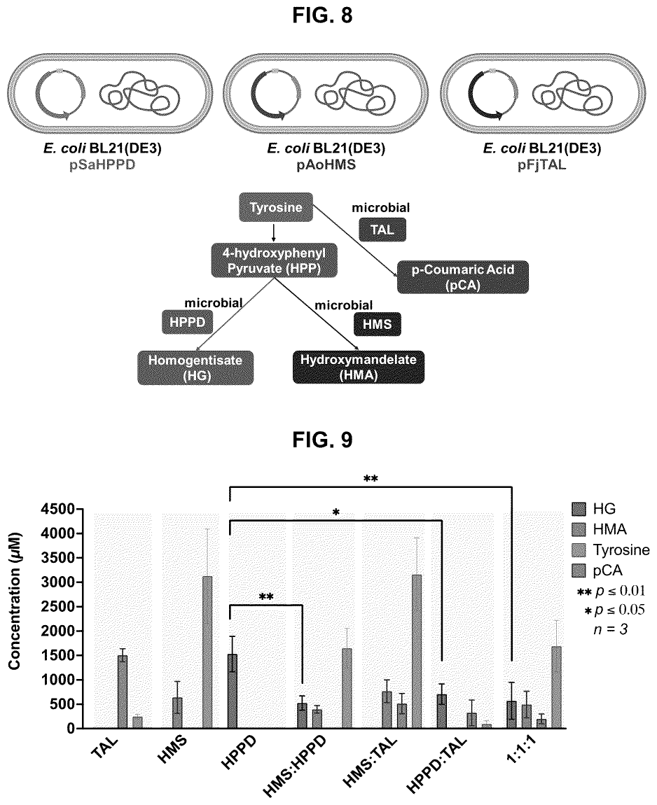

To further understand if the competitions in utilizing tyrosine are driven by enzyme activities and if such experiments can be helpful in predicting metabolic outcomes with complex microbial communities, we created a synthetic community with E. coli BL21 (DE3) pLysS harboring SaHPPD, E. coli BL21 (DE3) pLysS harboring AoHMS, and E. coli Rosetta (DE3) harboring FjTAL (FIG. 7 and FIG. 8). Flavobacterium johnsoniae is an environmental microbe similar to A. orientalis and S. avermitilis. F. johnsoniae produces yellow pigment by utilizing p-coumaric acid (pCA). pCA can be generated from tyrosine via the action of tyrosine ammonia lyase (TAL). In this community, tyrosine metabolic branching is occurring at two different points (FIG. 7 and FIG. 8). The first one is occurring at tyrosine, where FjTAL can pull it out of the homogentisate pathway and can directly convert it into p-coumaric acid (pCA) (FIG. 8). The second branching point is occurring at 4-HPP, where AoHMS can convert it into HMA (FIG. 8).

Spent media of various mixed cultures and pure cultures were analyzed to detect and measure metabolites of tyrosine breakdown (FIG. 9). Here, while comparing the concentration of HG produced by SaHPPD to HG produced by the mixed culture of 1:1 ratio of SaHPPD:AoHMS, there was a significant decrease in HG in the mixed culture compared to the pure culture with only SaHPPD (FIG. 9). Moreover, we found that HG produced by SaHPPD was slightly higher than HMA produced by AoHMS in this 1:1 mix (FIG. 9), indicating higher activity coming from HPPD. We also observed around more than 1.5 mM of tyrosine in the spent media of 1:1 mix of AoHMS and SaHPPD. Such increase in tyrosine concentration in the mixed cultures of AoHMS and SaHPPD was observed before in our two-enzyme competing system experiments.

Similarly, for the mixed culture of 1:1 ratio of SaHPPD:FjTAL, HG was decreased significantly in the mixed culture vs. pure culture. Analysis of the 1:1:1 mixture of SaHPPD:AoHMS:FjTAL showed the presence of almost equimolar concentrations of HG and HMA with two to three fold less pCA. In addition, the spent media showed the presence of almost 1.6 mM tyrosine. This mixture with the synthetic community of three different E. coli strains harboring three different enzymes, behaved very similarly to the 1:1 mix culture of HPPD:HMS. In this context, FjTAL did not provide much reduction of HG, especially while one of the goals was to decrease the amount of tyrosine going through the homogentisate pathway. Here we show that, AoHMS provided better metabolic diversion strategy if measured only by the change in HG levels. However, the huge increase seen in the tyrosine within the spent media of pure or mixed AoHMS cultures is not desirable. It will be interesting to understand if the increase in tyrosine is specific to E. coli cells especially in the presence of AoHMS.

### Competition Between a Human HPPD and Gut Microbial TAL Provides a Novel Therapeutic Strategy for Metabolic Disorders of Tyrosine Breakdown

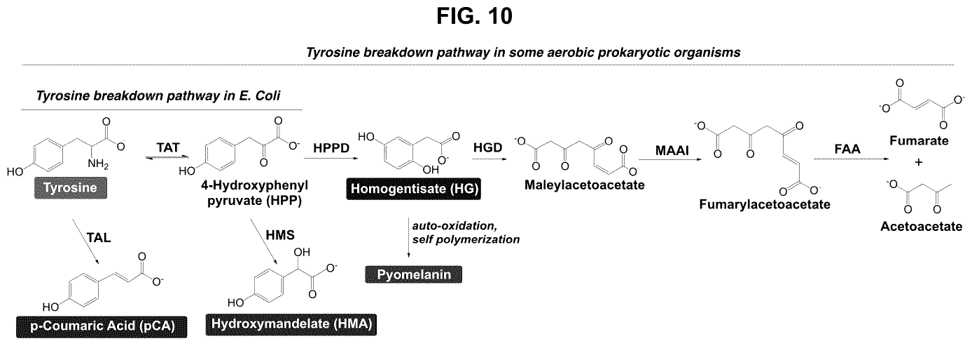

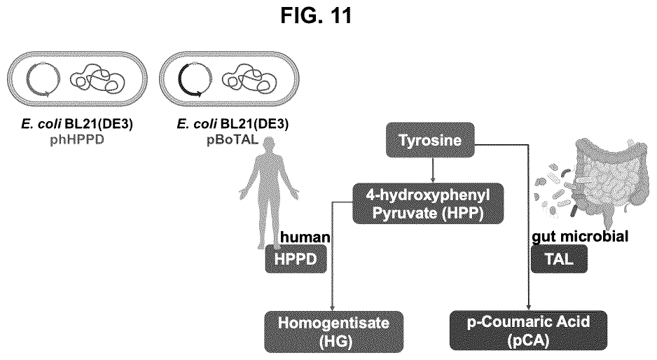

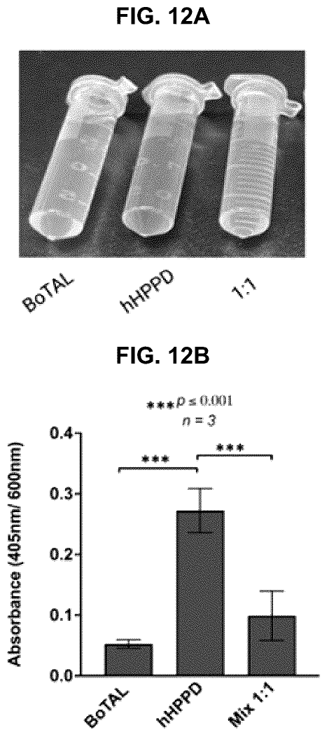

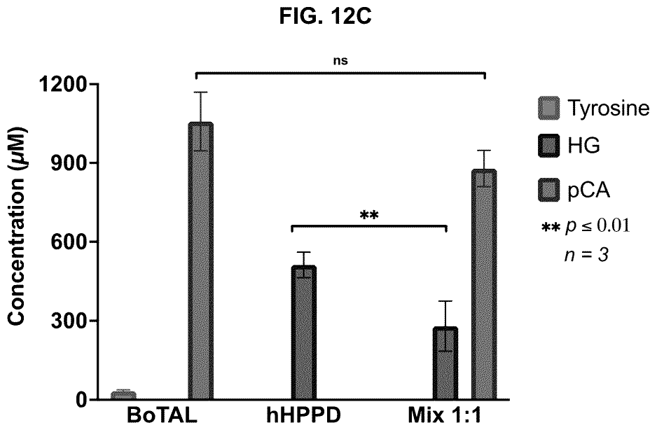

Disorders that arise from tyrosine breakdown pathway severely impact the life of a patient even with the FDA approved therapy. Although the medication given to these patients inhibits the tyrosine breakdown pathway, in the long run it can increase blood tyrosine concentrations which can lead to precipitation of tyrosine in the blood. For this reason, a therapeutic strategy where the tyrosine breakdown can be diverted from the mammalian homogentisate dependent pathway that stops the production of multiple harmful molecules under diseased conditions would provide a way to keep the blood tyrosine concentrations low (FIG. 10 and FIG. 11). To test this hypothesis, we expressed human HPPD enzyme (hHPPD), which catalyzes the conversion of 4-HPP to HG, and a tyrosine ammonia lyase (BoTAL) from the prominent gut microbe Bacteroides ovatus, which catalyzes the conversion of tyrosine to pCA, in E. coli BL21 (DE3) cells (FIG. 11). Our pigmentation assay showed that the metabolic diversion was possible when tyrosine was partitioned between these two metabolic pathways (FIG. 12). We observed a notable decrease in the production of the pigment when E. coli BL21 (DE3) strains containing hHPPD and BoTAL were mixed, which is a proxy for the decrease in the levels of secreted homogentisate (HG) (FIG. 2). This provided the first clue about the possibility of such metabolic diversions due to the competition between the activities of a human enzyme and a gut microbial enzyme.

Furthermore, the analysis of spent media provided additional information about the nature of this competition. We observed a marked decrease in the production of HG when cultures expressing hHPPD and BoTAL were mixed at 1:1 ratio (FIG. 12). Additionally, there was no tyrosine found in the spent media from the 1:1 mixed culture. These results show that, two basic requirements to create a potential therapeutic strategy were met here. The first where we detected a complete conversion of tyrosine during competition which provides a way to handle the insolubility of tyrosine. The second where due to the diversion from HG pathway to the pCA pathway, the concentrations of harmful metabolites produced via faulty tyrosine catabolism involving HG pathway would subsequently possibly be reduced.

Through this study, we show that the delicate balance of tyrosine metabolites is controlled by the competitive interactions within different metabolic pathways, which can possibly encompass both inter-microbial competition and the dynamic between microbes and their host. The contest for tyrosine can potentially impact microbial community structures in various environments, while a similar competition in the human gut may have implications for human health. This study has highlighted the complexity of tyrosine metabolism within synthetic microbial communities, emphasizing the influence of specific enzymatic activities. Furthermore, we have demonstrated a unique metabolic redirection of tyrosine degradation by leveraging a competition between a human enzyme and a gut microbial enzyme, suggesting a promising approach for the development of therapeutics in the future. This could possibly offer innovative treatments for metabolic disorders linked to tyrosine metabolism which will need to be experimentally tested.

What is claimed:

1. A method for analyzing competition for tyrosine breakdown, comprising:
providing a first microbial strain expressing a first enzyme that converts a tyrosine pathway intermediate to a first metabolite;
providing a second microbial strain expressing a second enzyme that converts the tyrosine pathway intermediate to a second metabolite different from the first metabolite;
optionally providing a third microbial strain expressing a third enzyme that converts tyrosine to a third metabolite different from the first metabolite and the second metabolite;
combining the first microbial strain, the second microbial strain, and the third microbial strain, if present, to form a synthetic microbial community;
culturing the synthetic microbial community in a medium comprising tyrosine, thereby creating competition between the first enzyme, the second enzyme, and the third enzyme, if present.

2. The method of claim 1, further comprising:
measuring a quantity of the first metabolite, the second metabolite, or the third metabolite, wherein the first enzyme, the second enzyme, and the third enzyme, if present, compete for the tyrosine pathway intermediate or tyrosine, thereby diverting tyrosine metabolism from a first pathway producing the first metabolite toward a second pathway producing the second metabolite or a third pathway producing the third metabolite.

3. The method of claim 1, wherein the first microbial strain, the second microbial strain, and the third microbial strain, if present, are Escherichia coli strains.

4. The method of claim 3, wherein the Escherichia coli strains are E. coli BL21 (DE3) strains.

5. The method of claim 1, wherein the tyrosine pathway intermediate is 4-hydroxyphenylpyruvate.

6. The method of claim 5, wherein the first enzyme is a 4-hydroxyphenylpyruvate dioxygenase that converts 4-hydroxyphenylpyruvate to homogentisate.

7. The method of claim 6, wherein the 4-hydroxyphenylpyruvate dioxygenase is from Streptomyces avermitilis or Homo sapiens.

8. The method of claim 5, wherein the second enzyme is a hydroxymandelate synthase that converts 4-hydroxyphenylpyruvate to hydroxymandelate.

9. The method of claim 8, wherein the hydroxymandelate synthase is from Amycolatopsis orientalis.

10. The method of claim 1, wherein the second enzyme is a tyrosine ammonia lyase that converts tyrosine to p-coumaric acid or the third enzyme is a tyrosine ammonia lyase that converts tyrosine to p-coumaric acid.

11. The method of claim 10, wherein the tyrosine ammonia lyase is from Flavobacterium johnsoniae.

12. The method of claim 10, wherein the tyrosine ammonia lyase is from Bacteroides ovatus.

13. The method of claim 1, wherein the first microbial strain and the second microbial strain are combined at a ratio between about 1:4 and about 4:1.

14. The method of claim 2, wherein measuring the quantity of the first metabolite, the second metabolite, or the third metabolite comprises:
centrifuging the medium to form a supernatant and a pellet; and
measuring an absorbance of the supernatant,
wherein the first metabolite is pyomelanin.

15. A composition comprising:
a first recombinant Escherichia coli strain comprising an expression vector encoding a 4-hydroxyphenylpyruvate dioxygenase capable of converting 4-hydroxyphenylpyruvate (HPP) into homogentisate (HG),
wherein the first recombinant Escherichia coli strain produces pyomelanin by secreting HG;
a second recombinant Escherichia coli strain comprising an expression vector encoding a second enzyme that converts a tyrosine pathway intermediate to a second metabolite; and optionally
a third recombinant Escherichia coli strain comprising an expression vector encoding a third enzyme that converts tyrosine to a third metabolite different from the first and second metabolites;
wherein the first recombinant Escherichia coli strain produces a quantity of pyomelanin inversely proportional to an activity of the second enzyme.

16. The composition of claim 15, wherein the first enzyme is a 4-hydroxyphenylpyruvate dioxygenase from Streptomyces avermitilis or a 4-hydroxyphenylpyruvate dioxygenase from Homo sapiens.

17. The composition of claim 15, wherein the second enzyme is a hydroxymandelate synthase from Amycolatopsis orientalis or a tyrosine ammonia lyase from Bacteroides ovatus.

18. The composition of claim 15, wherein the third enzyme is a tyrosine ammonia lyase from Flavobacterium johnsoniae.

19. The composition of claim 15, wherein the first recombinant Escherichia coli strain, the second recombinant Escherichia coli strain, and the third recombinant Escherichia coli strain, if present, are E. coli BL21 (DE3) strains.

20. The composition of claim 15, wherein the ratio of the first recombinant Escherichia coli strain to the second recombinant Escherichia coli strain is between about 1:4 and about 4:1.

21. A method for detecting a concentration of one or more tyrosine pathway intermediates in a sample, the method comprising:
administering to the sample one or more bacterial strains comprising an expression vector encoding an enzyme selected from the group consisting of:
a 4-hydroxyphenylpyruvate dioxygenase from Streptomyces avermitilis or from Homo sapiens;
a hydroxymandelate synthase from Amycolatopsis orientalis; and
a tyrosine ammonia lyase from Bacteroides ovatus or Flavobacterium johnsoniae;
centrifuging the sample to isolate a supernatant; and
measuring the absorbance of the supernatant to determine a concentration of pyomelanin.

22. The method of claim 21, wherein measuring the absorbance of the supernatant comprises analyzing the supernatant via HPLC.
